(19) United States
(12) Patent Application Publication
(10) Pub. No.: US 2024/0400533 A1
(43) Pub. Date: Dec. 5, 2024

(54) General Solid-Phase Synthesis of Crown Ethers

(71) Applicant: University of Windsor, Windsor (CA)

(72) Inventors: John F. TRANT, LaSalle (CA); Sarah Nasri, LaSalle (CA); John J. Hayward, Windsor (CA)

(21) Appl. No.: 18/678,589

(22) Filed: May 30, 2024

Related U.S. Application Data
(60) Provisional application No. 63/505,818, filed on Jun. 2, 2023.

(51) Int. Cl. C07D 323/00

(52) U.S. Cl. CPC C07D 323/00

(57) ABSTRACT

In a preferred embodiment, there is provided a method for preparing a crown ether, the method comprising: loading a resin having a resin functional group with a linking compound having a functional group selected for coupling to the resin functional group and at least two carbon atoms each having a substituent; coupling the linking compound to a polyethylene glycol having two terminal carbon atoms each bonded to an optionally protected terminal hydroxyl group to obtain a cyclized intermediate, whereby one said at least two carbon atoms or the substituent and an associated one of the two terminal carbon atoms or the hydroxyl groups undergo a coupling or substitution reaction; and removing the linking compound and the cyclized intermediate from the resin.

### FIELD OF THE INVENTION

This invention relates to a method for solid-phase synthesis of a crown ether, and which preferably includes cyclization of a polyethylene glycol on a resin with use of a linking compound therebetween.

### BACKGROUND OF THE INVENTION

Polymeric resins have been used as solid support for peptide synthesis and cyclized peptide rings. In peptide synthesis, the polymeric resins may be coupled through amide formation and cleaved irreversibly that requires disposal of resins or harsh conditions to replenish the lost function group.

By way of an example, aminomethyl polystyrene resins may be used as a starting material to be customized into a specific function group or act as an aldehyde scavenger in gel chromatography. Amine and aldehyde groups may form reversible imine bonds, which allows for cleaving of the bonds and restore amine function with mild conditions.

Previously, crown ethers have been obtained by solution-phase synthesis, which are often associated with unintended oligomerization of reactants and/or products, as well as inability or reduced yield for preparing crown ethers of larger sizes. For instance, a previous method for solution-phase synthesis of an 18-crown-6 includes the following reaction scheme and associated yields using standard conditions:

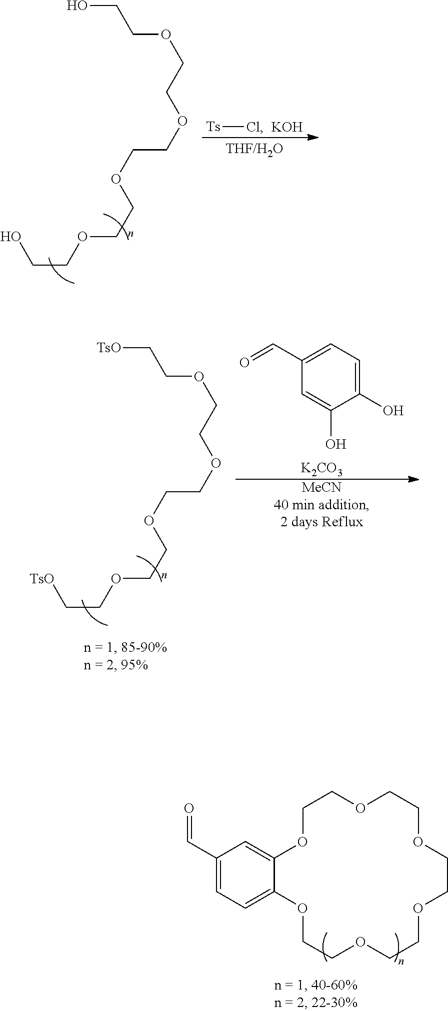

### SUMMARY OF THE INVENTION

A possible non-limiting object of the present invention is to provide a method for solid-phase synthesis of a crown ether, and which may permit synthesis on a solid support, thereby reducing unintended polymerization of reactants, intermediates and/or products.

Another possible non-limiting object of the present invention is to provide a method for solid-phase synthesis of a crown ether, and which may permit operation to prepare crown ethers of greater ring size.

Another possible non-limiting object of the present invention is to provide a method for solid-phase synthesis of a crown ether, and which may permit synthesis with immobilization of a reactant or intermediate on a solid support for subsequent removal therefrom, thereby reducing possible side reactions and thus improved yield.

In one simplified aspect, the present invention provides a method for preparing a cyclic product compound, the method comprising: loading a resin having a resin functional group with a linking compound having a functional group selected for coupling to the resin functional group and at least two carbon atoms each having a substituent; coupling the linking compound to a reactant compound having a linear portion provided with two terminal carbon atoms each bonded to an optionally protected terminal hydroxyl group to obtain a cyclized intermediate, whereby one said at least two carbon atoms or the substituent and an associated one of the two terminal carbon atoms or the hydroxyl groups undergo a coupling or substitution reaction; and removing the linking compound and the cyclized intermediate from the resin.

In another simplified aspect, the present invention provides a method for preparing a crown ether, the method comprising: loading a resin having a resin functional group with a linking compound having a functional group selected for coupling to the resin functional group and at least two carbon atoms each having a substituent; coupling the linking compound to a polyethylene glycol having two terminal carbon atoms each bonded to an optionally protected terminal hydroxyl group to obtain a cyclized intermediate, whereby one said at least two carbon atoms or the substituent and an associated one of the two terminal carbon atoms or the hydroxyl groups undergo a coupling or substitution reaction; and removing the linking compound and the cyclized intermediate from the resin.

In one embodiment, the at least two carbon atoms are electrophilic, and the substitution reaction comprises substitution of the substituents of the at least two carbon atoms by the hydroxyl groups. In an alternative embodiment, the substituents are nucleophilic, and the substitution reaction comprises substitution of the hydroxyl groups by the substituents.

In one aspect, the present invention provides a method for preparing a crown ether, the method comprising: loading a resin having a resin functional group with a linking compound having a functional group selected for coupling to the resin functional group and at least two adjacent carbon atoms each substituted with a nucleophilic substituent; coupling the linking compound to a polyethylene glycol having two terminal carbon atoms each bonded to an optionally protected terminal hydroxyl group, whereby each said nucleophilic substituent is coupled to an associated one of the two terminal carbon atoms to obtain a cyclized intermediate; and removing the linking compound and the cyclized intermediate from the resin.

In another aspect, the present invention provides a method for preparing a crown ether, the method comprising: loading a resin having an amino functional group with a linking compound having a carbonyl selected for forming an imine bond with the amino functional group, the linking compound further comprising at least two carbon atoms each having an optionally protected hydroxyl group; coupling the linking compound to a polyether polyol having two terminal carbon atoms each bonded to an optionally protected hydroxyl group to obtain a cyclized intermediate, whereby each said at least two carbon atoms forms an ether bond with an associated one of the two terminal carbon atoms; and removing the cyclized intermediate from the resin to obtain the crown ether.

In yet another aspect, the present invention provides a method for preparing a crown ether, the method comprising: loading a resin having a resin functional group with a linking compound having a functional group selected for coupling to the resin functional group and at least two carbon atoms each having an optionally protected hydroxyl group; coupling the linking compound to a polyether polyol having two terminal carbon atoms each bonded to an optionally protected hydroxyl group to obtain a cyclized intermediate, whereby each said at least two carbon atoms forms an ether bond with an associated one of the two terminal carbon atoms; and removing the cyclized intermediate from the resin to obtain the crown ether.

In yet another aspect, the present invention provides a method for preparing a crown ether, the method comprising: loading a resin having a resin functional group with a linking compound having a functional group selected for coupling to the resin functional group and at least two carbon atoms each substituted with a first substituent; coupling the linking compound to a reactant compound of the formula X—CH₂—CH₂—(O—CH₂—CH₂)ₙ—X, wherein n is an integer between 1 and 17, and X is a second substituent, one of the first and second substituents being a leaving group and a remaining one of the first and second substituents being a nucleophilic substituent, whereby the linking compound and the reactant compound undergo a substitution reaction with the nucleophilic substituent replacing the leaving group to thereby obtain a cyclized intermediate; and removing the cyclized intermediate from the resin to obtain the crown ether.

It is to be appreciated that the resin is not particularly limited, provided that the resin contains the resin functional group operable to react with the functional group of the linking compound and permits for synthesis of the crown ether thereon. In one embodiment the resin is a polystyrene-based resin, a controlled pore-glass bead, a polyethylene oxide based resin, a 2-acrylamidoprop-1-yl-(2-aminoprop-1-yl) polyethylene glycol based resin, or a polycaprolactone-based resins. In one embodiment, the resin is (aminomethyl) polystyrene resin, Amberlite XAD-4 resin, Amberlite FPA66 resin or aminomethyl polystyrene resin.

In one embodiment, the resin functional group is amino, an aldehyde, formyl, a thiol, a furan or a maleimide. In one embodiment, the resin is functionalized with amine, hydrazine, ketone or aldehyde. In one embodiment, loading or conjugation to the resin is through conjugation, and removing the linking compound and the cyclized intermediate from the resin or cleavage from the resin is through hydrolysis.

In one embodiment, the resin comprises an aminomethylpolystyrene resin, optionally wherein the aminomethylpolystyrene resin is crushed. In one embodiment, the resin functional group is an amino functional group, an aminomethyl functional group, a hydrazinyl functional group, a carbonylmethyl functional group, a hydroxymethyl functional group, a methanethiol functional group, an ethane-1,2-diol functional group, a maleimide functional group or a furanyl functional group. In one embodiment, the functional group comprises a carbonyl, carboxyl, thiol, ketone, aldehyde, amine, hydrazine, boronic acid or maleimide.

In one embodiment, the crown ether is optionally substituted 18-crown-6, 21-crown-7, 24-crown-8, 27-crown-9, 30-crown-10, 33-crown-11, 36-crown-12, 39-crown-13, 42-crown-14, 45-crown-15 or a derivative thereof. It is to be appreciated that the method is not strictly restricted to include coupling the linking compound to the ethylene glycol only, and rather, the linking compound may be coupled to one or more other compounds having two hydroxyl groups or other reactive or functional groups, such as, but not limited, to polypropylene glycol, polybutylene glycol or a derivative thereof. In another embodiment, the crown ether has between 10 and 60, between 12 and 55, between 14 and 50 or between 18 and 45 ring atoms made up of monomers derived from one or more of optionally substituted ethylene glycol, propylene glycol and butylene glycol.

In one embodiment, the crown ether has a structural formula as identified below:

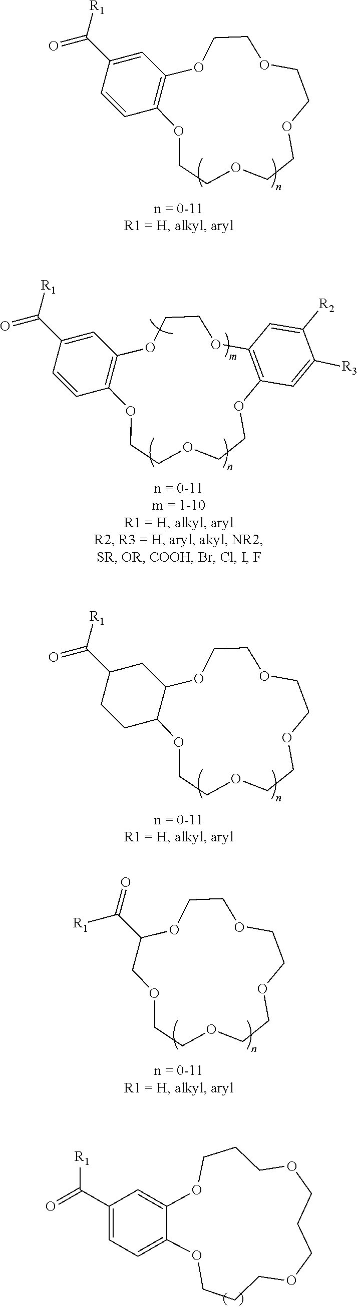

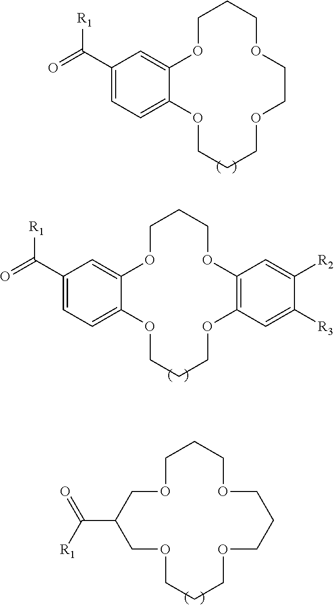

It has been envisioned that for larger crown ethers (24C8 and bigger), 3,4-dihydroxybenzaldehyde, or another 1,2, 1,3 or 1,4-diol may be reacted with chloroalkyl alcohol or bromoalkyl alcohol oligomers of ethylene glycol, propylene glycol, or butylene glycol.

It is to be appreciated that the polyethylene glycol is not strictly limited and may be selected based on the ring size of the intended crown ether. In one embodiment, the polyethylene glycol is pentaethylene glycol or hexaethylene glycol. In one embodiment, the polyethylene glycol comprises tosylated, mesylated or triflated terminal hydroxyl groups. In an alternative embodiment, one or both of the terminal hydroxyl groups are replaced with a halogen, preferably bromine, iodine, or chlorine. In one embodiment, the terminal hydroxyl groups are replaced by a leaving group, wherein the leaving group comprises tosylate, mesylate, bromide, iodide, triflate or chloride. In one embodiment, the leaving group comprises dinitrogen, dialkyl ether, halogen, hydroxyl, nitrate, phosphate, thioether, amino, alkoxy, carboxylate, phenoxide, alkoxyl, amido, acyloxy (preferably —OAc, —OC(O)CF₃), sulfonate (preferably mesyl or tosyl), acetamide (preferably —NHC(O)Me), carbamate (preferably N(Me)C(O)Ot-Bu), phosphonate (preferably —OP(O)(OEt)₂) or alcohol.

In one embodiment, the polyethylene glycol has the chemical formula H—(O—CH₂—CH₂)ₙ—OH, wherein n is an integer between 2 and 18, and terminal hydroxyl groups of the polyethylene glycol are optionally protected. In one embodiment, n is an integer between 5 and 9. In one embodiment, n is an integer between 4 and 8.

In one embodiment, the linking compound comprises substituted aryl, heteroaryl, cycloalkyl or heterocycloalkyl. In one embodiment, the linking compound comprises substituted phenyl or naphthalenyl. In one embodiment, the linking compound comprises 1,2-dihydroxynaphthalene. In one embodiment, the linking compound comprises catechol substituted with the functional group. In one embodiment, the linking compound is 3,4-dihydroxybenzaldehyde. In an alternative embodiment, the substituent or the nucleophilic substituent are coupled to carbon atoms not adjacent to each other. In another embodiment, the linking compound is glyceraldehyde or 3-amino-propane diol.

In one embodiment, the linking compound comprises a substituted aryl or heteroaryl compound. In one embodiment, the at least two carbon atoms form two carbon ring atoms of the substituted aryl or heteroaryl compound. In one embodiment, the substituted aryl or heteroaryl compound comprises a benzene compound, the functional group being located para to the optionally protected hydroxyl group or the first substituent of one said two carbon ring atoms. In one embodiment, the at least two carbon atoms form two adjacent carbon ring atoms of the substituted aryl or heteroaryl compound, the polyether polyol comprising polyethylene glycol. In one embodiment, the linking compound comprises a benzene compound, the at least two carbon atoms forming two carbon ring atoms of the benzene compound. In one embodiment, the carbonyl forms part of a formyl bonded to the benzene compound, the formyl being located para to the optionally protected hydroxyl group of one said two carbon ring atoms. In one embodiment, the at least two carbon atoms form two adjacent carbon ring atoms of the benzene compound, the polyether polyol comprising polyethylene glycol. In one embodiment, the linking compound is 3,4-dihydroxybenzaldehyde.

It is to be appreciated that the combination of the resin functional group and the functional group of the linking compound are not particularly limited, provided that the groups allow a reaction to load the linking compound to the resin. Likewise, the combination of the substituent or nucleophilic substituent of the linking compound and the terminal carbon atoms of the polyethylene glycol are not particularly limited, provided that the substituent and the terminal carbon atoms permit a reaction for cyclization of the polyethylene glycol.

In one embodiment, the hydroxyl groups of the at least two carbon atoms are unprotected, and the hydroxyl groups of the two terminal carbon atoms are protected, optionally wherein the hydroxyl groups of the two terminal carbon atoms are protected with methanesulfonyl or toluenesulfonyl. In an alternative embodiment, the hydroxyl groups of the at least two carbon atoms are protected, and the hydroxyl groups of the two terminal carbon atoms are unprotected, optionally wherein the hydroxyl groups of the at least two carbon atoms are protected with methanesulfonyl or toluenesulfonyl.

In one embodiment, the first substituent is the nucleophilic substituent, and the second substituent is the leaving group. In an alternative embodiment, the first substituent is the leaving group, and the second substituent is the nucleophilic substituent. In one embodiment, the leaving group is selected from the group consisting of tosylate, mesylate, bromide, iodide, triflate and chloride. In one embodiment, the nucleophilic substituent is hydroxyl.

In one embodiment, said coupling the linking compound to the polyether polyol or the reactant compound is performed with a base, optionally wherein the base is K₂CO₃.

In one embodiment, the term “amino” refers to a functional group having a nitrogen atom bonded to two hydrogen atoms, where one or both of the hydrogen atoms may optionally be substituted, preferably but not limited to, alkyl or aryl, i.e., the amino includes primary, secondary, tertiary or quaternary amino. For instance, the amino includes alkylamino, dialkylamino or trialkylamino.

In one embodiment, the term “cycloalkyl” refers to a hydrocarbon 3-8 membered monocyclic or 7-14 membered bicyclic ring system having at least one non-aromatic ring. The Cycloalkyl group is optionally substituted with one or more substituents, and may be cyclopropyl, cyclopentyl, cyclohexyl, cyclobutyl, cycloheptyl, cyclooctyl, cyclononyl or cyclodecyl.

In one embodiment, the term “heterocycloalkyl” refers to a nonaromatic 5-8 membered monocyclic, 8-12 membered bicyclic or 11-14 membered tricyclic ring system comprising 1-3 heteroatoms if monocyclic, 1-6 heteroatoms if bicyclic or 1-9 heteroatoms if tricyclic, said heteroatoms being O, N, S, B, P or Si. The heterocycloalkyl is optionally substituted with one or more substituents. In one embodiment, the heterocycloalkyl is piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, tetrahydrothienyl or thiirene.

In one embodiment, the term “aryl” refers to a hydrocarbon monocyclic, bicyclic or tricyclic aromatic ring system, and which is optionally substituted with one or more substituents. In one embodiment, the aryl is phenyl, naphthyl, anthracenyl, fluorenyl, indenyl or azulenyl.

In one embodiment, the term “heteroaryl” refers to an aromatic 5-8 membered monocyclic, 8-12 membered bicyclic or 11-14 membered tricyclic ring system having 1-4 ring heteroatoms if monocyclic, 1-6 heteroatoms if bicyclic or 1-9 heteroatoms if tricyclic, where the heteroatoms are independently O, N or S, and the remainder ring atoms are carbon. The heteroaryl is optionally substituted with one or more substituents. In one embodiment, the heteroaryl is pyridyl, 1-oxo-pyridyl, furanyl, benzo[1,3]dioxolyl, benzo[1,4]clioxinyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, and benzo[b]thienyl, 3H-thiazolo[2,3-c][1,2,4]thiadiazolyl, imidazo[1,2-d]-1,2,4-thiadiazolyl, imidazo[2,1-b]-1,3,4-thiadiazolyl, 1H,2H-furo[3,4-d]-1,2,3-thiadiazolyl, 1H-pyrazolo[5,1-c]-1,2,4-triazolyl, pyrrolo[3,4-d]-1,2,3-triazolyl, cyclopentatriazolyl or pyrrolo[2,1b]oxazolyl.

In one embodiment, the term “substituent” or “substituted” means that a hydrogen radical is replaced with a group that does not substantially adversely affect the stability or activity of the compound. The term “substituted” refers to one or more substituents, which may be the same or different, each replacing a hydrogen atom. In one embodiment, the substituent is halogen, hydroxyl, amino, alkylamino, arylamino, dialkylamino, diarylamino, cyano, nitro, mercapto, oxo, carbonyl, thio, imino, formyl, carbamido, carbamyl, carboxyl, thioureido, thiocyanato, sulfoamido, sulfonylalkyl, sulfonylaryl, alkyl, alkenyl, alkoxy, mercaptoalkoxy, aryl, heteroaryl, cyclyl, heterocyclyl, wherein alkyl, alkenyl, alkyloxy, aryl, heteroaryl, cyclyl and heterocyclyl are optionally substituted with alkyl, aryl, heteroaryl, halogen, hydroxyl, amino, mercapto, cyano, nitro, oxo, thioxo or imino.

Any and all compounds identified or described herein may be optionally substituted with a substituent, unless explicitly indicated to the contrary.

Additional and alternative features of the present invention will be apparent to a person skilled in the art from the following detailed description of the preferred embodiments thereof.

### BRIEF DESCRIPTION OF THE DRAWINGS

Reference may now be had to the following detailed description taken together with the accompanying drawings in which:

FIG. 1 shows photographs of test vials in a ninhydrin test performed after the first trial of removals, where vial C is commercial control; vial 1 used HCl exclusively; vial 2 NH₄OH exclusively; and vial 3 used HCl then NH4OH, and where the photograph on the right shows the vials after 5 minutes, in which vials C and 3 turned purple while vials 1 and 2 remained yellow;

FIG. 2 shows an outline of reversible imine and related linkages to immobilize the linking compound to the resin, where R, R1 and R2 represent scaffolds attached to diols;

FIG. 3 shows condensation-related alternatives for the immobilization of linkers to the resin, where R, R1, and R2 represent scaffolds attached to diols;

FIG. 4 shows furan-maleimide Diels-Alder reaction for reversible conjugation of a linking compound or crown ether substrate to the resin;

FIG. 5 shows a ¹H NMR spectrum of (ethane-1,2-diylbis(oxy))bis(ethane-2,1-diyl)bis(4-methylbenzenesulfonate) (1c);

FIG. 6 shows a ¹H NMR spectrum of ((oxybis(ethane-2,1-diyl))bis(oxy))bis(ethane-2,1-diyl)bis(4-methylbenzenesulfonate) (1d);

FIG. 7 shows a ¹H NMR spectrum of 3,6,9,12-tetraoxatetradecane-1,14-diyl bis(4-methylbenzenesulfonate) (1e);

FIG. 8 shows a ¹H NMR spectrum of 3,6,9,12,15-pentaoxaheptadecane-1,17-diyl bis(4-methylbenzenesulfonate) (1f);

FIG. 9 shows a ¹³C NMR spectrum of 3,6,9,12,15-pentaoxaheptadecane-1,17-diyl bis(4-methylbenzenesulfonate) (1f);

FIG. 10 shows a ¹H NMR spectrum of 3,6,9,12-tetraoxatetradecane-1,14-diyl dimethanesulfonate (2e);

FIG. 11 shows a ¹H NMR spectrum of on-resin synthesized 4′-Formylbenzo[18C6] following purification;

FIG. 12 shows an IR spectrum of on-resin synthesized 4′-Formylbenzo[18C6] following purification;

FIG. 13 shows a ¹H NMR spectrum of on-resin synthesized 4′-Formylbenzo[21C7] following purification;

FIG. 14 shows IR spectra of commercial amine-functionalized polystyrene resin;

FIG. 15 shows IR spectra of 4-(PS-iminomethyl)benzene-1,2-diol;

FIG. 16 shows IR spectra of (3,4-dimethoxybenzylidene)methanamine-PS;

FIG. 17 shows comparative IR spectra, where commercial resin was fresh, unused aminomethyl-PS, loaded SM was 4-(PS-iminomethyl)benzene-1,2-diol, and loaded crown ether was 4-(PS-iminomethyl) 4′-formylbenzo 18C6 crown ether, loaded from solution-prepared 4′-Formylbenzo[18C6] on to resin using same loaded method as SM; This figure is meant to provide fingerprinting for the user for comparing synthetic batches with prior to cleavage from the resin.

FIG. 18 shows a ¹H NMR spectrum of on-resin synthesized 4′-Formylbenzo[18C6] directly removed from resin crude with no workup or isolation other than partial removal of solvent;

FIG. 19 shows ¹H NMR spectrum of dimethoxybenzyl model;

FIG. 20 shows a ¹H NMR spectrum of 2,2′-(1,2-phenylenebis(oxy))bis(ethan-1-ol) (A);

FIG. 21 shows a ¹³C NMR spectrum of 2,2′-(1,2-phenylenebis(oxy))bis(ethan-1-ol) (A);

FIG. 22 shows ¹H NMR spectra of 4′-formylbenzo[24C8];

FIG. 23 shows a ¹H NMR spectrum of 4′-formylbenzo[27C9]; and

FIG. 24 shows a ¹H NMR spectra of 4′-formylbenzo[30C10].

### DETAILED DESCRIPTION OF THE PREFERRED EMBODIMENTS

In a preferred non-limiting embodiment, the method was performed in accordance with the following reaction pathway:

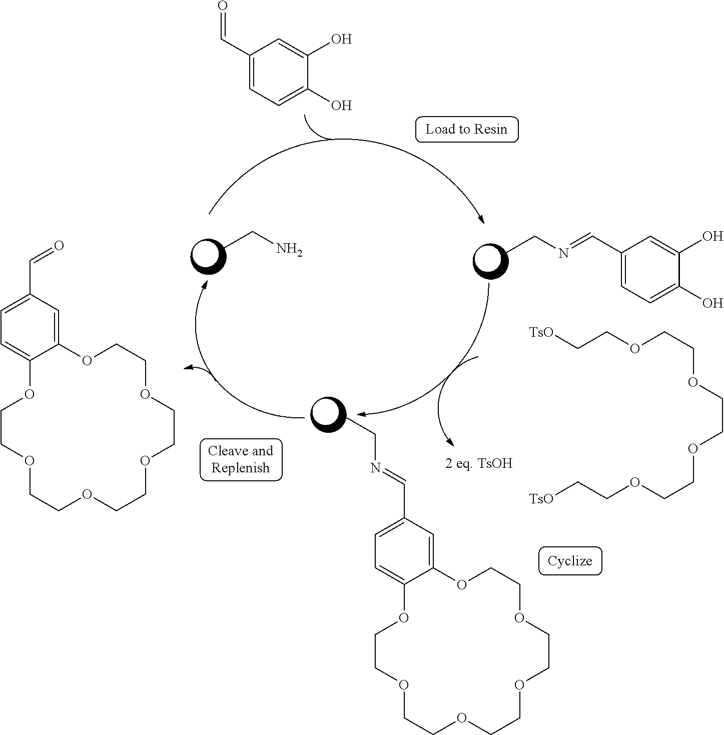

Specifically, the reaction pathway as shown above includes initial loading of an aldehyde substituted aromatic base onto a resin having an amino or primary amino functional group, such as an aminomethylpolystyrene resin, through an imine formation. The reaction pathway further includes introducing cyclization conditions, such as those previously established by solution chemistry or solution-phase synthesis, to produce a cyclized intermediate on the resin support and then releasing the cyclized intermediate to obtain a benzocrown ether under acidic conditions. The resin, which would contain a terminal ammonium would be then replenished back into amine by pH adjustment to restore reactivity, so as to permit reuse in future reactions.

The applicant has appreciated that the method may permit more ready and convenient preparation of a crown ether on a solid support, allowing for separation of resin beads from a reaction mixture and removing or washing away of excess reagents, while maintaining the intended product until cleavage from the resin, while reducing polymerization of reactants, intermediates and/or products at least in part by immobilization of the intermediates to promote intramolecular ring closure and not the polymerization.

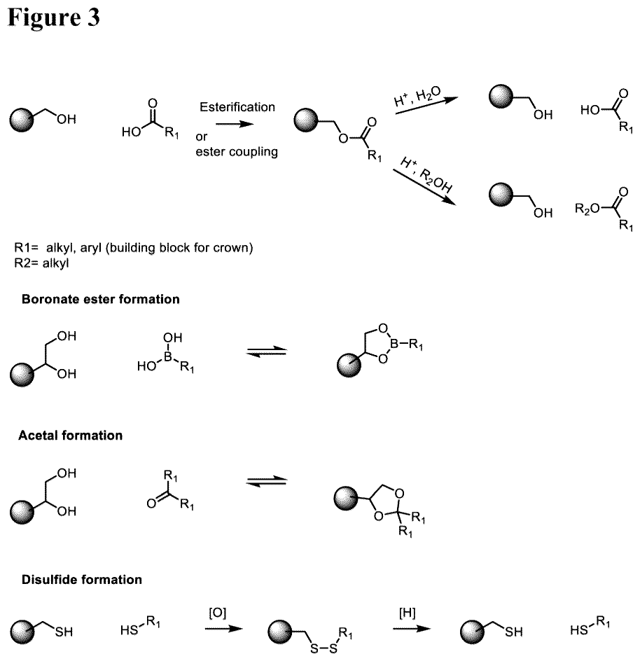

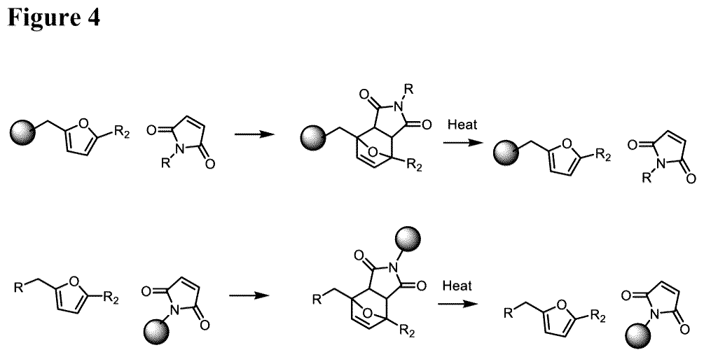

Although the above reaction pathway shows resin loading by imine formation, it is to be appreciated that the loading may be performed with other functional groups and/or reactions. FIG. 2 shows other reactions which may occur during the loading step, with, for example, an aminomethyl resin (top), a hydrazine resin (middle) and a carbonylmethyl resin (bottom). The loading step may be performed with other resins, functional groups and reactions, permitting for coupling between the resin functional group and the functional group of the linking compound. By way of non-limiting examples, and as seen in FIG. 3, such other functional groups and reactions may include esterification, boronate esters, acetals, or disulfides, which may use condensation and related reactions to generate stable intermediates that can be reversed with water and acid or, in the case of disulfides, with reducing conditions. As further seen in FIG. 4, the ligand may be loaded or conjugated to the resin using a reversible Diels-Alder reaction between a furan and maleimide.

### Reagents and Instruments

Commercially available regents used were sourced from Sigma Aldrich, otherwise stated, which includes methylamine polystyrene resin (70-90 mesh and 200-400 mesh 1-1.5 g/mol loading), 3,4-dihydroxybenzyaldehyde, 3,4-dimethoxybenzaldehyde, and Tosylated chloride. Peg chains were sourced from Oakwood Chemicals ACS grade acetonitrile, ethyl acetate, tetrahydrofuran and other chemicals were purchased from Sigma-Aldrich, AK Scientific, Oakwood Chemicals, Alfa Aesar or Acros Chemicals. Reagents were used without prior purification. All heated reactions were conducted using oil baths or metal bead baths on IKA RET Basic stir plates equipped with a P1000 temperature probe. All reactions under elevated temperatures used all glass joints in contact with the solvent. Septa were generally used at the top of condensers. Vacuum and gases were introduced from a Schlenk line using needles through septa during reactions, and generally directly using glass adapters during drying of samples in round bottom flasks. Thin layer chromatography was performed using EMD aluminum-backed silica 60 F254-coated plates and were visualized using either UV-light (254 nm), KMnO₄, vanillin, Hanessian's stain, PMA, or DNP stain. Standard work-up procedure for all reactions undergoing an aqueous wash, unless otherwise stated, involved back extraction of every aqueous phase, a drying of the combined organic phases with anhydrous magnesium sulphate, filtration either using vacuum and a sintered-glass frit or through a glass-wool plug using gravity, and concentration under reduced pressure on a rotary evaporator (Büchi or Synthware). Depending on the compound, various methods were used to purify titled compounds. The exact conditions used would be mentioned in synthetic procedure. ¹H NMR spectra were obtained at 300 MHz or 500 MHz, and proton-decoupled, and ¹³C NMR spectra were obtained at 75 or 125 MHz on Bruker instruments. NMR chemical shifts (δ) are reported in ppm and are calibrated against residual solvent signals of CHCl₃ (δ7.26), DMSO-d₆ (δ 2.54), acetone-d₅ (δ 2.05), or methanol-d₃ (δ 3.31). Proton NMR was acquired for all crude mixtures prior to purification even when not otherwise stated. LRMS ESI (positive and negative) was run by Advion ESI LR Mass and processed data by Advion MassExpress and Advion DataExpress. HRMS were conducted on Thermo Scientific Orbitrap Velos Pro (Easy-nLC/HESI Hybrid Ion Trap-Orbitrap Mass Spectrometer) by Queens University (Canada). IR was run on Bruker IR using solid loading and measured between 4000 to 400 cm⁻¹. Pictures were taken directly from the software or processed through Microsoft Excel.

### Polyethylene Glycol

While the reaction pathway as identified above is shown with a cyclization reaction involving tosylated polyethylene glycol, it is to be appreciated that the cyclization reaction may be conducted with unprotected polyethylene glycol or polyethylene glycol protected with other protecting groups, such as mesylate. In an alternative embodiment, terminal hydroxyl groups of polyethylene glycol may be replaced with other leaving groups, such as a halogen.

Provided below is a reaction scheme showing three different methods A to C for converting a polyethylene glycol to include different leaving groups:

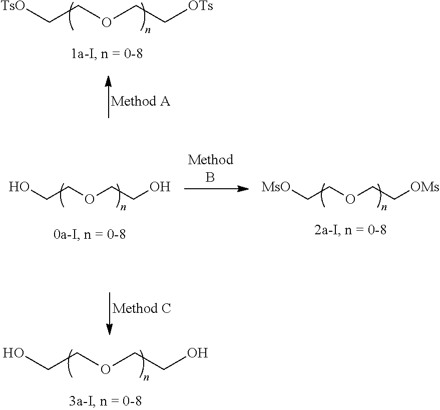

General Method A. Tosylation of PEG components: The method may follow procedure based on previous literature or Y. Chen and G. L. Baker, The Journal of Organic Chemistry, 1999, 64, 6870-6873. PEG (42 mmol) and 4-toluenesulfonyl chloride (24.1 g, 126 mmol) were dissolved in THF (110 ml) and cooled down to 0° C. To the mixture, pestle-and-mortar crushed KOH (14 g, 252 mmol) was dissolved in H₂O (16 ml) and the solution was slowly poured added and stirred for overnight at room temperature. Upon completion, THF was evaporated, and H₂O was added followed by an extraction with diethyl ether (2×). After separating the layers, the organic layer was washed with brine then dried with MgSO₄. The solvent was evaporated to obtain a crude.

By way of selected example of general method A, the following compounds were prepared:

### (ethane-1,2-diylbis(oxy))bis(ethane-2,1-diyl)bis(4-methylbenzenesulfonate) 1c

Following Method A, the Sample was a White Solid. Yield %: 79% Over 1 Reaction.

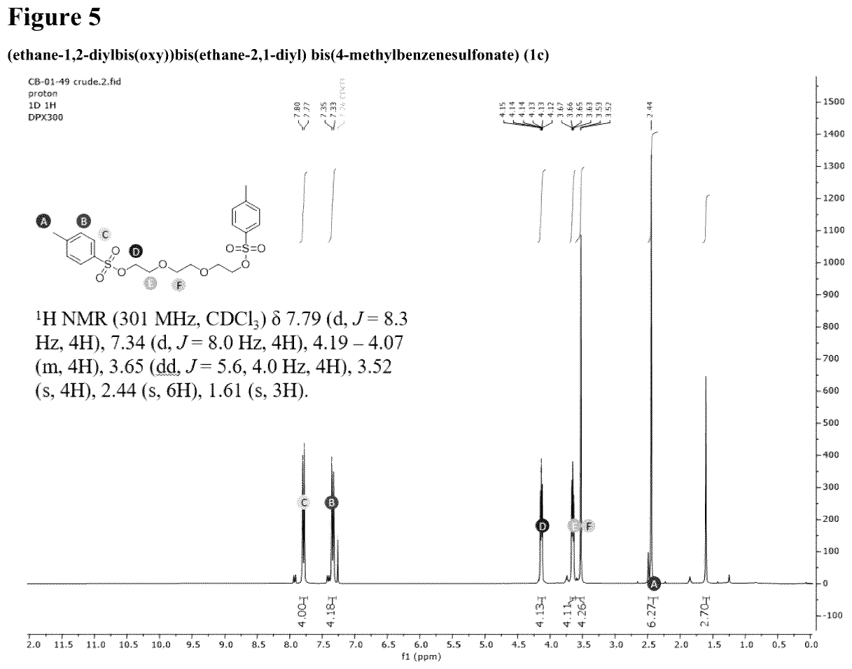

¹H NMR (301 MHZ, CDCl₃) δ 7.79 (d, J=8.3 Hz, 4H), 7.34 (d, J=8.0 Hz, 4H), 4.19-4.07 (m, 4H), 3.65 (dd, J=5.6, 4.0 Hz, 4H), 3.52 (s, 4H), 2.44 (s, 6H), 1.61 (s, 3H). See FIG. 5.

HRMS (ESI) [M+H]⁺ calc'd C₂₀H₂₇O₈S₂+: 459.11419 (predicted) found: 459.11383.

### ((oxybis(ethane-2,1-diyl))bis(oxy))bis(ethane-2,1-diyl)bis(4-methylbenzenesulfonate) 1d

Following Method A, the Sample was a Clear Oil. Yield: 96%. Rf: 0.33 in 2 Hexane: 1 DCM: 1 Acetone

¹H NMR (300 MHz, CDCl₃) δ 7.85-7.68 (m, 4H), 7.50-7.30 (m, 4H), 4.15 (td, J=5.0, 1.3 Hz, 4H), 3.73-3.64 (m, 4H), 3.56 (dd, J=3.7, 1.4 Hz, 8H), 2.45 (d, J=6.2 Hz, 6H).

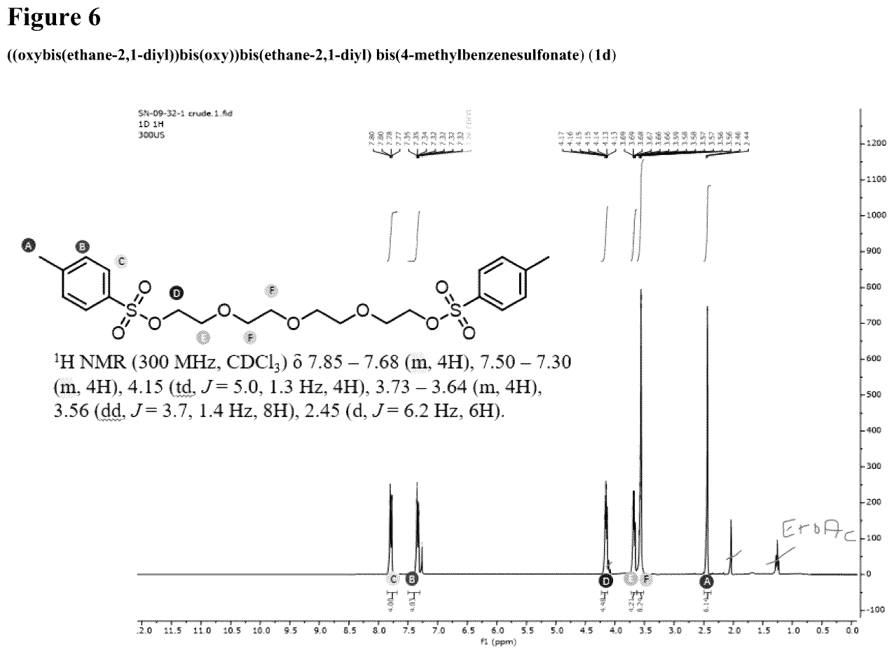

See FIG. 6. Similar to reported literature of title compound.

### 3,6,9,12-tetraoxatetradecane-1,14-diyl bis(4-methylbenzenesulfonate) 1e

Following Method A, the Sample is a White Solid. Yield %: 77%-90% Yield Over 3 Reactions. Rf: 0.52 in 2 Hexane: 1 DCM: 1 Acetone m.p.: 43-45° C.

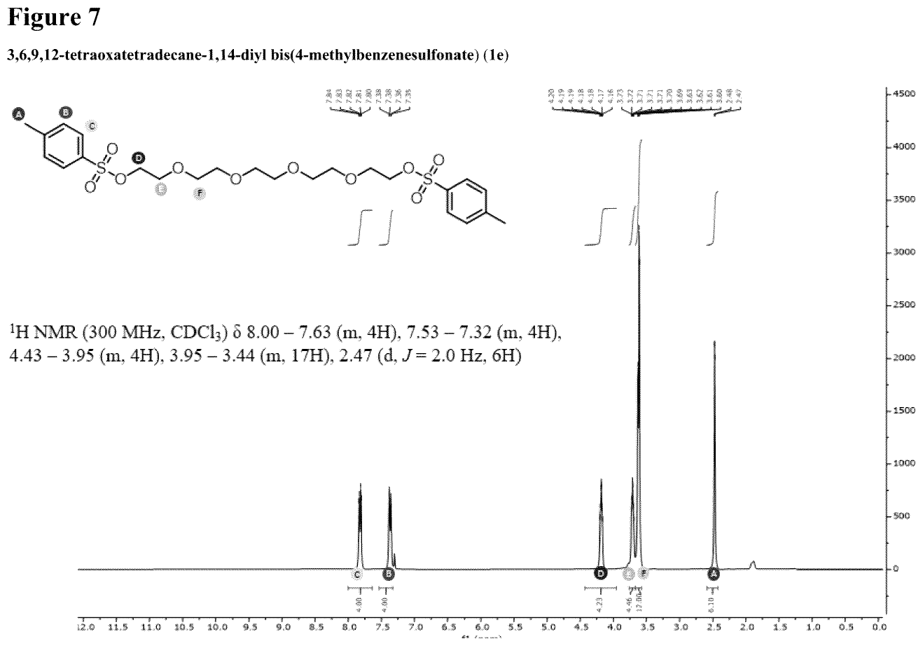

¹H NMR (300 MHz, CDCl3) δ 7.82-7.73 (d, 4H), 7.32 (d, J=8.1 Hz, 4H), 4.18-4.09 (m, 4H), 3.71-3.62 (m, 4H), 3.57 (d, J=6.6 Hz, 12H), 2.43 (s, 6H). See FIG. 7.

### 3,6,9,12,15-pentaoxaheptadecane-1,17-diyl bis(4-methylbenzenesulfonate) 1f

Following Method A, the Sample is a Thick Slightly Opaque Off-White Oil. Yield %: 95% Yield Over 1 Reaction. Rf: 0.36 in 2 Hexane: 1 DCM: 1 Acetone

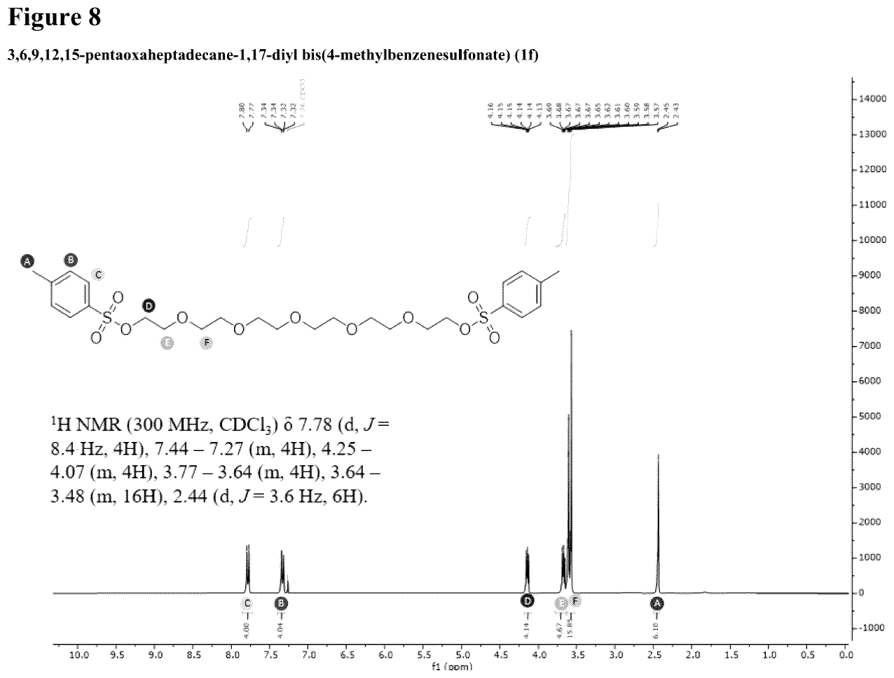

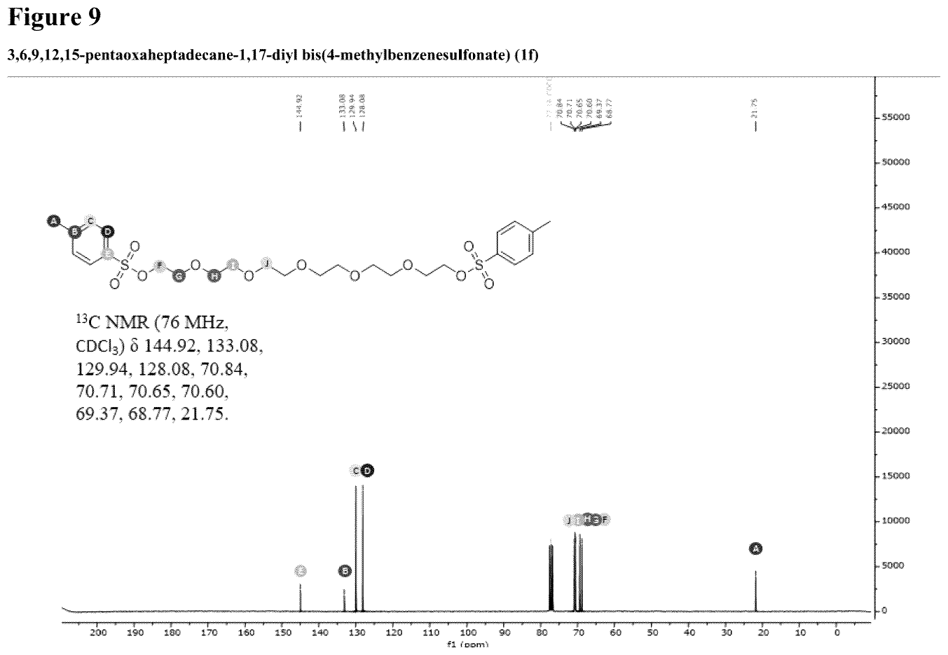

¹H NMR (300 MHz, CDCl₃) δ 7.78 (d, J=8.4 Hz, 4H), 7.44-7.27 (m, 4H), 4.25-4.07 (m, 4H), 3.77-3.64 (m, 4H), 3.64-3.48 (m, 16H), 2.44 (d, J=3.6 Hz, 6H). ¹³C NMR (76 MHz, CDCl₃) δ 144.92, 133.08, 129.94, 128.08, 70.84, 70.71, 70.65, 70.60, 69.37, 68.77, 21.75. See FIGS. 8 and 9.

General Method B. Mesylation of PEG components: In a flamed dried flask, PEG (4.3 mmol) and triethylamine (1.8 ml, 12.9 mmol) were dissolved into DCM (21 ml) at 0° C. Mesyl chloride (1 ml, 8.6 mmol) was added dropwise and left stirring at 0° C. for two hours. The solution was washed with 1M HCl (1×10 ml), sat. NaHCO₃ (1×10 ml), and brine (1×10 ml). The organic layer was dried by Mg₂SO₄ and evaporated by vacuo to afford a crude without further purification and was stored in −20° C. freezer to preserve it.

By way of selected example of general method B, the following compounds were prepared:

### 3,6,9,12-tetraoxatetradecane-1,14-diyl dimethanesulfonate 2e

Following Method B, the Sample was a Clear Oil that Slowly Crashed into Colorless Crystals. R_{f}: 0.30 in 2 Hexane: 1 DCM: 1 Acetone.

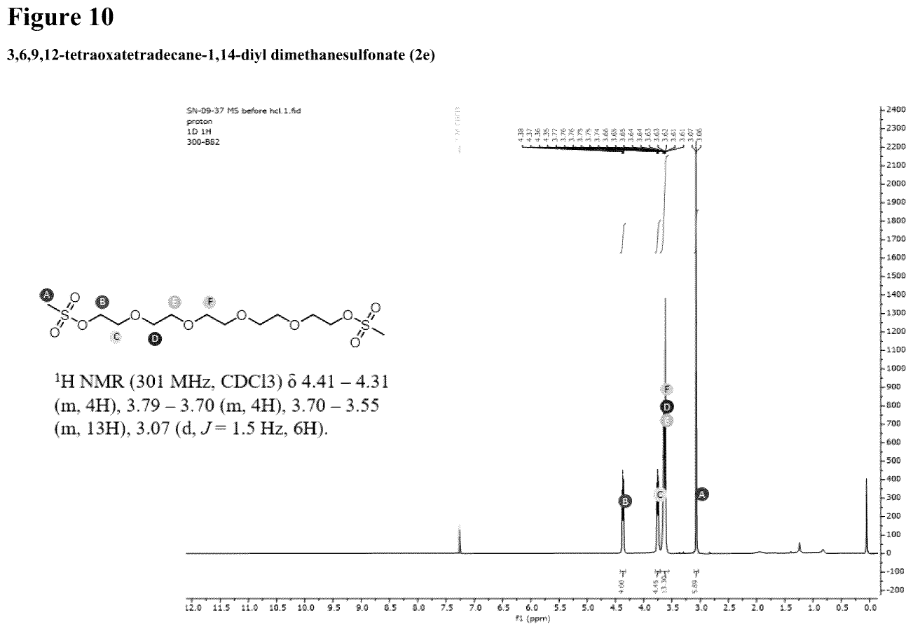

¹H NMR (301 MHz, CDCl3) δ 4.41-4.31 (m, 4H), 3.79-3.70 (m, 4H), 3.70-3.55 (m, 12H), 3.07 (d, J=1.5 Hz, 6H). ESI HR [M+H]⁺ calc'd C₁₂H₂₇O₁₀S₂⁺ for 395.10401 predicted, 395.10306 g/mol found. See FIG. 10.

General procedure for dimesylation of polyethylene glycol as a diol may include the following: The diol (5.0 g, 1.0 eq) was dissolved in CH₂Cl₂ (100 mL) and cooled to 0° C. under a nitrogen atmosphere. Triethylamine (2.6 eq) was diluted with CH₂Cl₂ (25 mL) and added to the solution of the diol. To the stirred solution, a solution of methanesulfonyl chloride (2.2 eq) in CH₂Cl₂ (25 mL) was added to the reaction mixture at 0° C., dropwise via an addition funnel. After completion of the addition, the reaction mixture was allowed to warm to room temperature over 2 h. A 5% aq HCl solution was added, and the mixture was stirred for 15 min. The biphasic mixture was decanted into a separatory funnel and the organic layer was separated. The organic solution was washed with saturated aq. NaHCO₃ (50 mL), distilled H₂O (50 mL) and brine (50 mL). The solution was dried over MgSO₄ and the solvent was removed in vacuo. Solid products were recrystallized from MeOH, and the products, which were oils at room temperature, were used without purification after their characterization by ¹H NMR and IR spectroscopy.

Furthermore, as an example of dimesylation of polyethylene glycol, 3,6,9,12-tetraoxatetradecane-1,14-diyl dimethanesulfonate was prepared as follows: In a flamed dried flask, pentaethylene glycol (1 g, 4.3 mmol) and triethylamine (1.8 ml, 12.9 mmol) were dissolved into DCM (21 ml) at 0° C. Mesyl chloride (1 ml, 8.6 mmol) was added dropwise and left stirring at 0° C. for two hours. The solution was washed with 1M HCl (1×10 ml), sat. NaHCO₃ (1×10 ml), and brine (1×10 ml). The organic layer was dried by Mg₂SO₄ and evaporated by vacuo to afford titled compound as a clear oil that slowly crashed into colorless crystals. The crude oil was used without further purification and was stored in −20° C. freezer to preserve it. Rf: 0.30 in 2 hexane: 1 DCM: 1 acetone. ¹H NMR (301 MHz, CDCl₃) δ 4.38-4.35 (m, 4H), 3.87-3.56 (m, 18H 16H?), 3.07 (s? d, J=0.8 Hz, 6H). ESI HR [M+H]⁺=295.10306 g/mol (395.104 predicted).

To test synthesis of tosylated polyethylene glycol, ethane-1,2-diyl bis(4-methylbenzenesulfonate) was prepared as follows: A 100 mL round bottom flask equipped with a stir bar was charged with ethylene glycol (0.62 g, 10 mmol), triethylamine (NEt₃, 3.34 mL, 24 mmol), ptosylchloride (TsCl, 4.58 g, 24 mmol), 4-dimethylaminopyridine (DMAP, 0.12 g, 1 mmol) and 20 mL anhydrous DCM. The reaction mixture was stirred overnight at RT. After reaction, 20 mL H₂O was added and the solution was extracted with DCM for three times. The combined organic layers were dried over anhydrous Na₂SO₄, concentrated under vacuum. The obtained light yellow solid was rinsed with methanol to give target compound 1,2-bis(p-tolylsulfonyloxy) ethane as a white solid (3.62 g, 98%). ¹H NMR (400 MHZ, CDCl₃) δ: 7.75 (d, J=8.4 Hz, 4H), 7.36 (d, J=8.4 Hz, 4H), 4.20 (s, 4H), 2.48 (s, 6H). ppm. ¹³C NMR (100 MHZ, CDCl₃) δ: 145.4, 132.3, 130.0, 127.9, 66.7, 21.7 ppm.

Furthermore, 3,6,9,12-tetraoxatetradecane-1,14-diyl bis(4-methylbenzenesulfonate) was prepared as follows: Pentaethylene glycol (10 g, 42 mmol) and 4-toluenesulfonyl chloride (24.1 g, 126 mmol) were dissolved in THF (110 ml) and cooled down to 0° C. To the mixture, pestle-and-mortar crushed KOH (14 g, 252 mmol) was dissolved in H₂O (16 ml) and the solution was slowly poured added and stirred for overnight at room temperature. Upon completion, H₂O was added then extracted with ether (2×). After separating the layers, the organic layer was washed with brine then dried with MgSO₄. The solvent was evaporated to obtain a yellow oil crude that slowly crashed out as pure white solid. There were no further purification. Yield %: 77%-90% yield over 3 reactions. Rf: 0.52 in 2 Hexane: 1 DCM: 1 Acetone m.p.: 43-45° C. ¹H NMR (300 MHz, CDCl₃) δ 7.82-7.73 (d, 4H), 7.32 (d, J=8.1 Hz, 4H), 4.18-4.09 (m, 4H), 3.71-3.62 (m, 4H), 3.57 (d, J=6.6 Hz, 12H), 2.43 (s, 6H).

In addition, 3,6,9,12,15-pentaoxaheptadecane-1,17-diyl bis(4-methylbenzenesulfonate) was prepared as follows: Hexaethylene glycol (10 g, 35.4 mmol) and 4-toluenesulfonyl chloride (20.3 g, 106 mmol) were dissolved in THF (110 ml) and cooled down to 0° C. To the mixture, pestle-and-mortar crushed KOH (11.9 g, 56 mmol) was dissolved in H₂O (13 ml) and the solution was slowly poured added and stirred for overnight at room temperature. Upon completion, H₂O was added then extracted with ether (2×). After separating the layers, the organic layer was washed with brine then dried with MgSO₄. The solvent was evaporated to obtain a thick slightly opaque clear oil crude. There were no further purification. Yield %: 95% yield Rf: 0.36 in 2 Hexane: 1 DCM: 1 Acetone ¹H NMR (300 MHz, CDCl₃) δ 7.78 (d, J=8.4 Hz, 4H), 7.44-7.27 (m, 4H), 4.25-4.07 (m, 4H), 3.77-3.64 (m, 4H), 3.64-3.48 (m, 16H), 2.44 (d, J=3.6 Hz, 6H).

Pertaining to an embodiment in which terminal hydroxyl groups of a polyethylene glycol is replaced by other leaving groups, 1,14-dichloro-3,6,9,12-tetraoxatetradecane was prepared as follows: 20.0 g (71 mmol) of hexaethylene glycol, 13 mL (179 mmol) of thionyl chloride, 0.1 mL of DMF and 40 mL of dry heptane was heated to reflux for 2 h in a 250 mL round bottom flask fitted with a stirrer and a condenser. Heptane and excess thionyl chloride were removed by distillation, to yield a viscous, yellowish oil. The product was vacuum distilled to render a pure colorless liquid. Yield: 90%. ¹H NMR (DMSO): d=3.51 (broad s, 16H); 3.54 (s, 4H); 3.68 (s, 4H). ¹³C NMR (DMSO): d=43.26; 69.61; 70.40.

Resin method: An example of the method of the invention was conducted in accordance with the following reaction scheme:

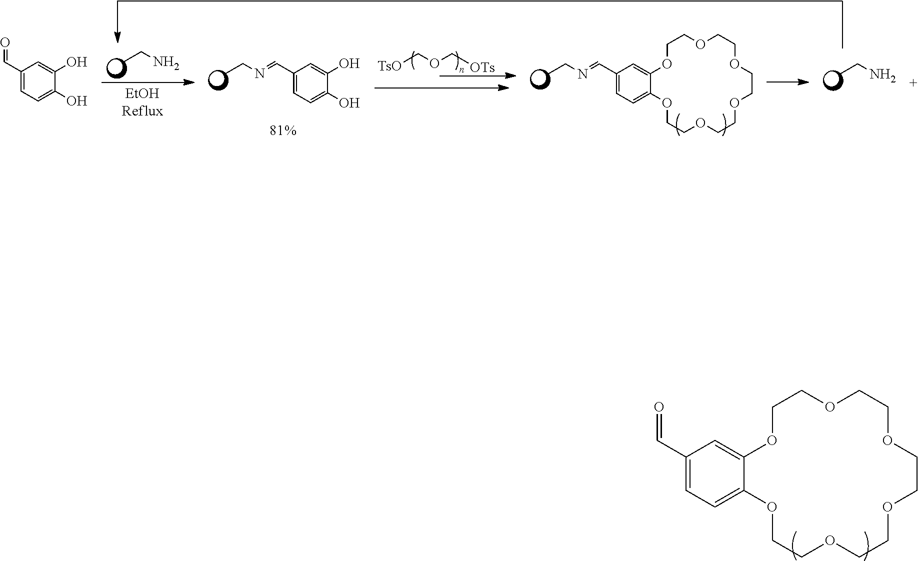

A reaction scheme similar to a solution method was performed on a solid support.

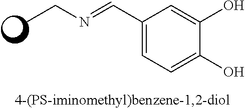

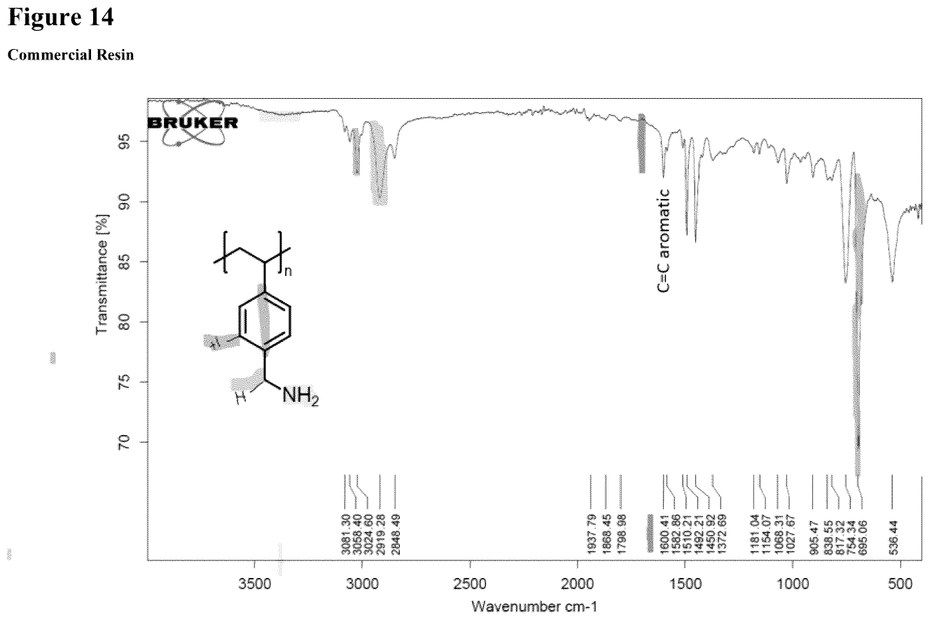

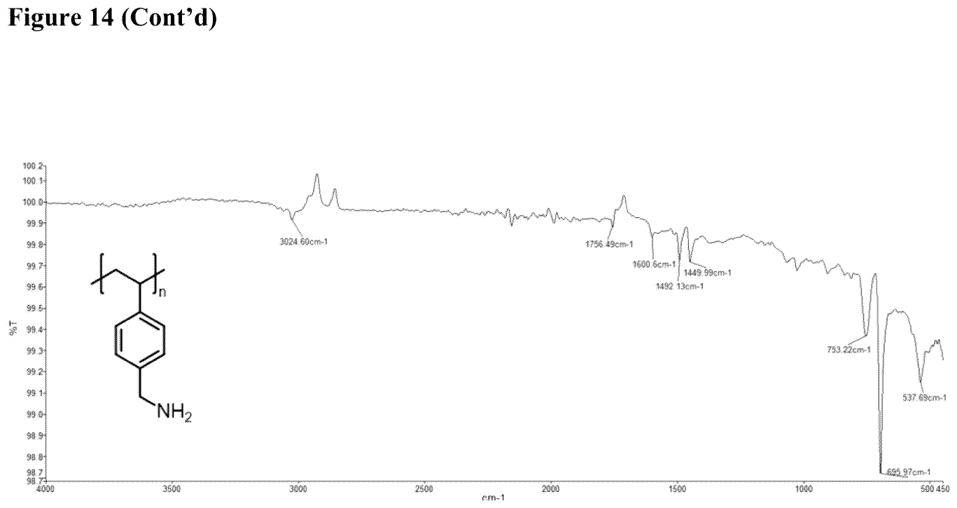

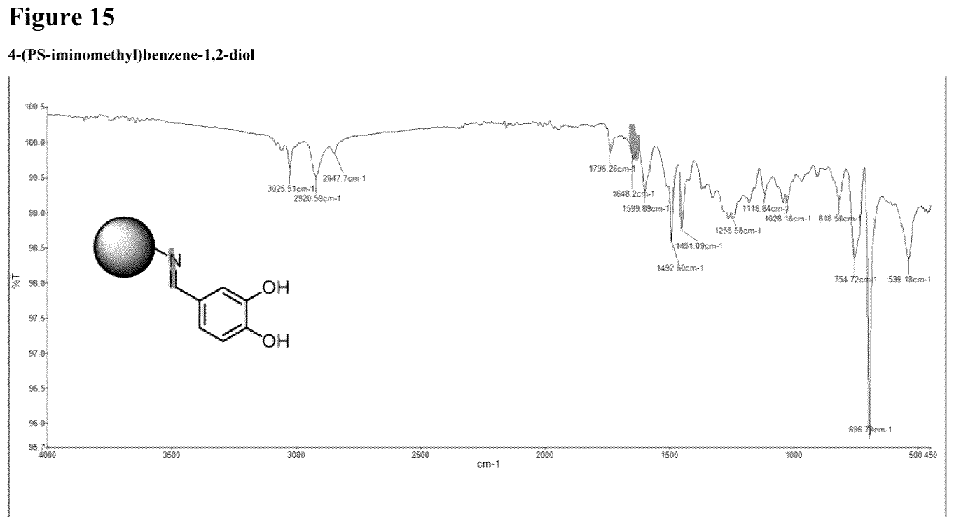

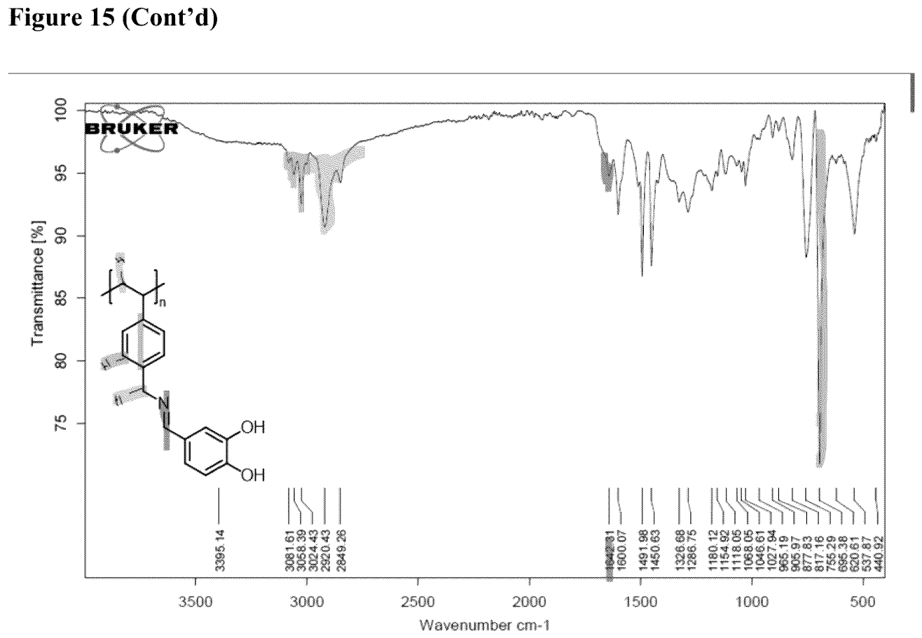

Either crushed (by pestle and mortar) for 5 minutes, or used directly from the bottle, aminomethyl PS resin (1.0-1.5 mol, 1 g) was mixed with 3,4-dihydroxybenzladehyde (2 mmol, 0.27 g) in ethanol under reflux conditions and nitrogen atmosphere for 3 h The red-brown solid was collected by filtration, washed with ethanol and DCM and dried under vacuum. A test for free amine was tested by taking a sample in a vial and putting two drops of ninhydrin and letting it stand for 15 minutes alongside of a vial of amidomethyl as a control. Any purple color development would indicate some free amine is still present and needs to be re-subjected to reaction conditions. No further purification was performed and continued to next reactions. General mass increase indicated mol loading was within the range stated of the product and exact mol/g of the batch was noted to calculate for further reactions. Resin-bound material was characterized by IR to confirm loading and conjugation (FIG. 15) compared to the base resin (FIG. 14).
It is to be appreciated that other solvents, such as THF, 2-THF, Toluene, DCM, DMF and others alike may be used in the loading step.

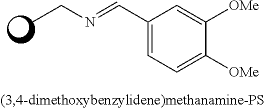

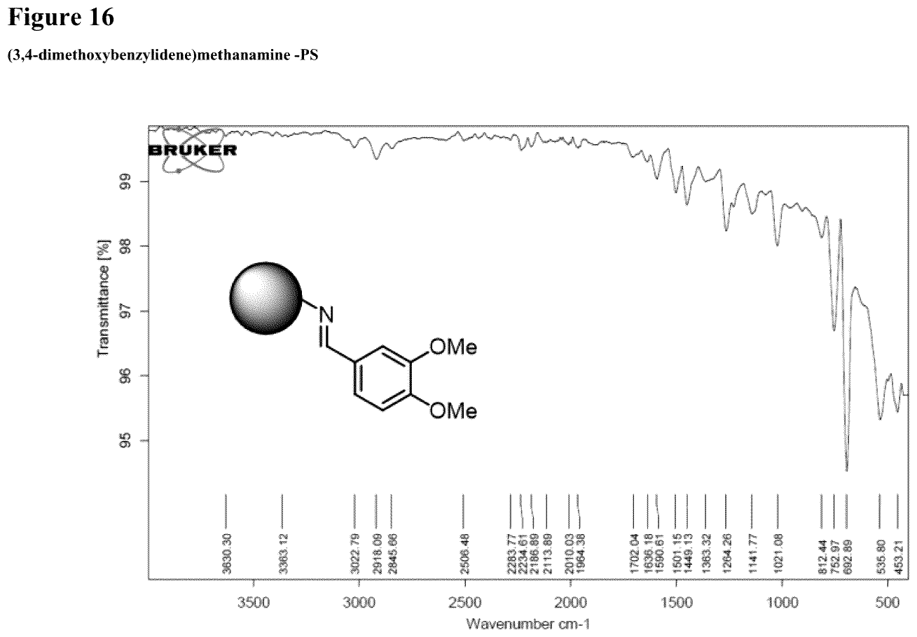

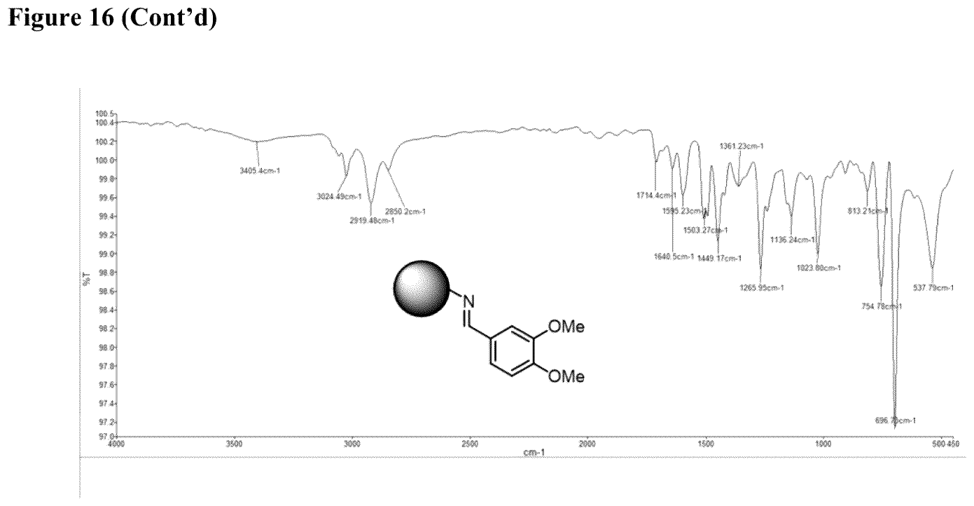

Aminomethyl PS resin (1.0-1.5 mol, 1 g) was mixed with 3,4-dimethoxybenzladehyde (2 mmol) in ethanol under reflux conditions and nitrogen atmosphere for 3 h. The yellow resin was collected by filtration, washed with ethanol and DCM and dried under vacuum. No further purification was performed and continued to next reactions. General mass increase indicated mol loading was within the range stated of the product and exact mol/g of the batch was noted to calculate for further reactions. Resin bound material was characterized by IR (FIG. 16)

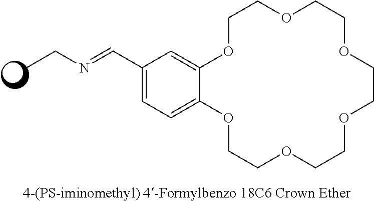 [SMILES: CC/N=C/c1ccc2c(c1)OCCOCCOCCOCCOCCO2]

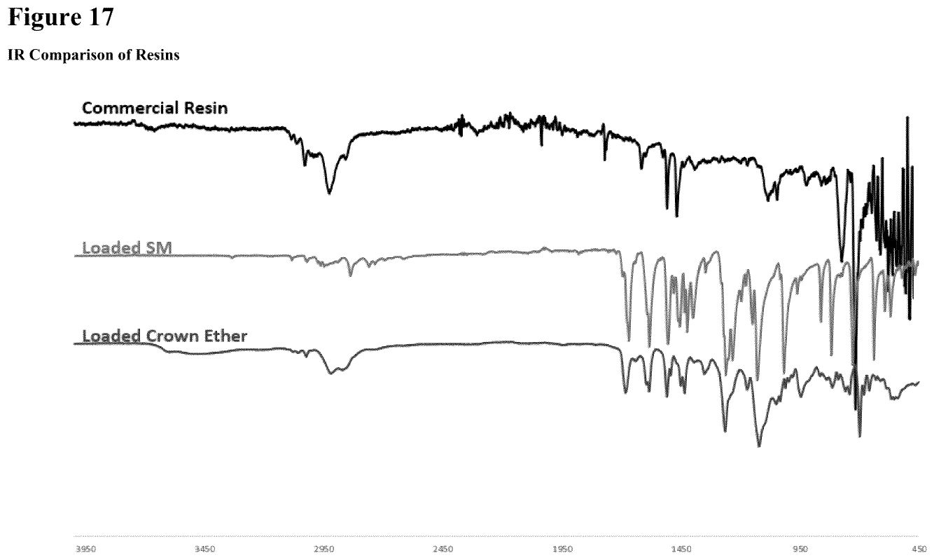

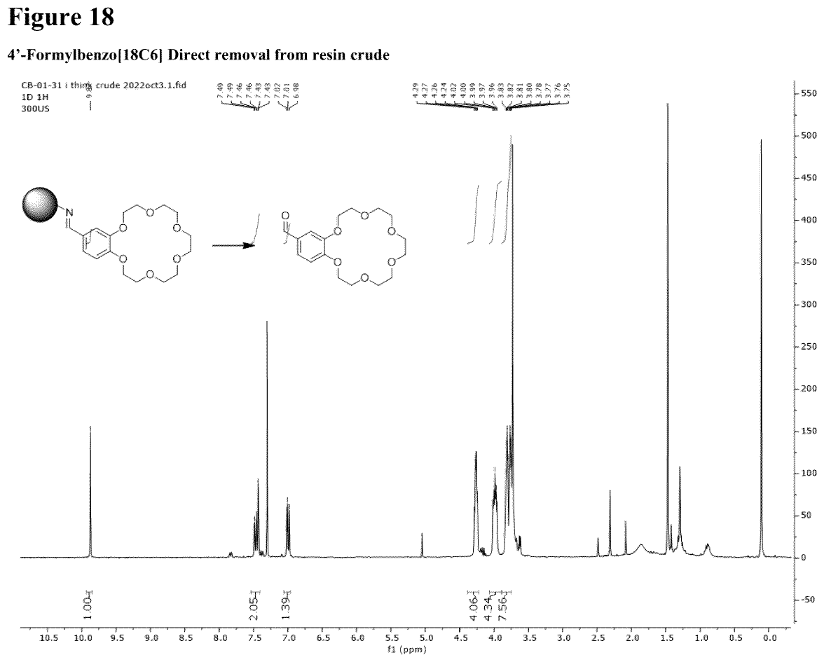

To a round bottom flask was charged-(PS-iminomethyl)benzene-1,2-diol (0.2 mmol loaded material), PEG (0.22 mmol), base (0.6 mmol) in organic solvent (10 ml). The reaction was refluxed for two days under Argon gas. Afterwards, the reaction was halted and the liquid was decanted carefully while keeping the resin in the flask. Washing of the resin began with sonification with DI water (5 ml×2) to remove salt then fresh THF (5 ml×3), decanting in between and then finally filtered to dry, IR of crude resin suggested conversion (i.e. FIG. 17). The crown ether was cleaved according to the above described procedure, and concentrated to dryness. The mass of the solid was weighed along with an NMR to identify conversion (i.e. FIG. 18). Table 1 features some conditions:

TABLE 1

| Base | Resin ^{a} | Solvent | Conversion ^{b} |
|---|---|---|---|
| K₂CO₃ | noncrushed | MeCN | 88% |
| K₂CO₃ | crushed | MeCN | 96% |
| K₂CO₃ | noncrushed | THF | 89% |

^{a} “Noncrushed” refers to using loaded resin that used resin directly from the bottle. “Crushed” refers to loaded resin that used resin that were crushed for 5 minutes in a pestle and mortar prior to loading step.
^{b} Conversion was determined by NMR through integration ratio of products and unreacted 3,4-dihydroxybenzaldehyde in acetone-d₆.

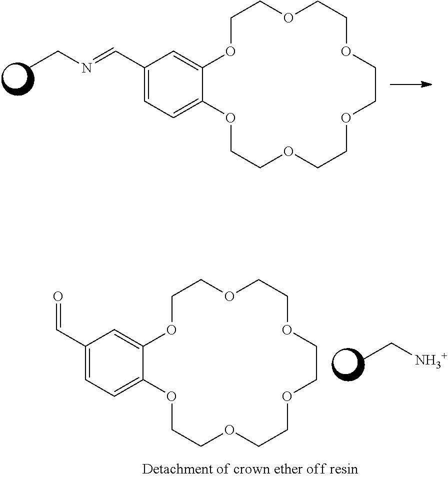

The resin was placed into a 4:1 THF: 1M HCl solution and agitated by a sonicator for 45 minutes. The resulting yellow solution was decanted, and the resin was sonicated briefly with fresh THE two more times or until solution ran clear. The THF in the solution was evaporated by vaccou and the remaining liquid is extracted by ethyl acetate (3×). The organic layer was combined and washed with brine (1×), then dried with magnesium sulfate, filtered, and concentrated. Further purification may be included, depending on the product crown ether.

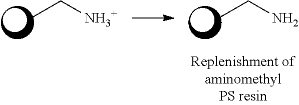

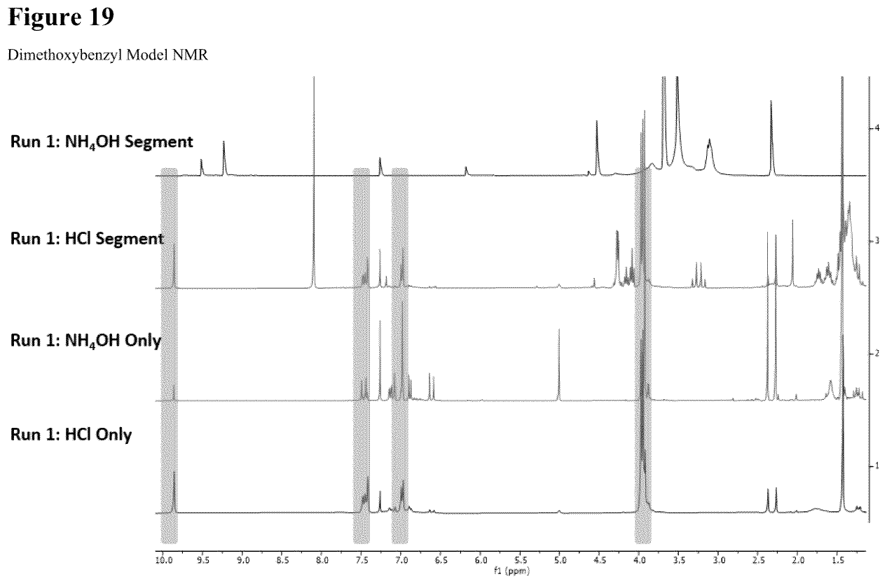

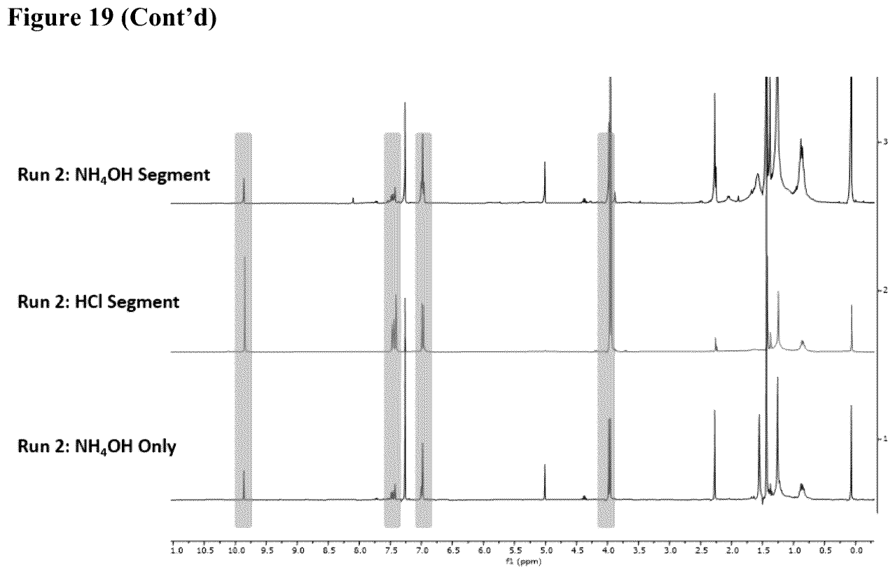

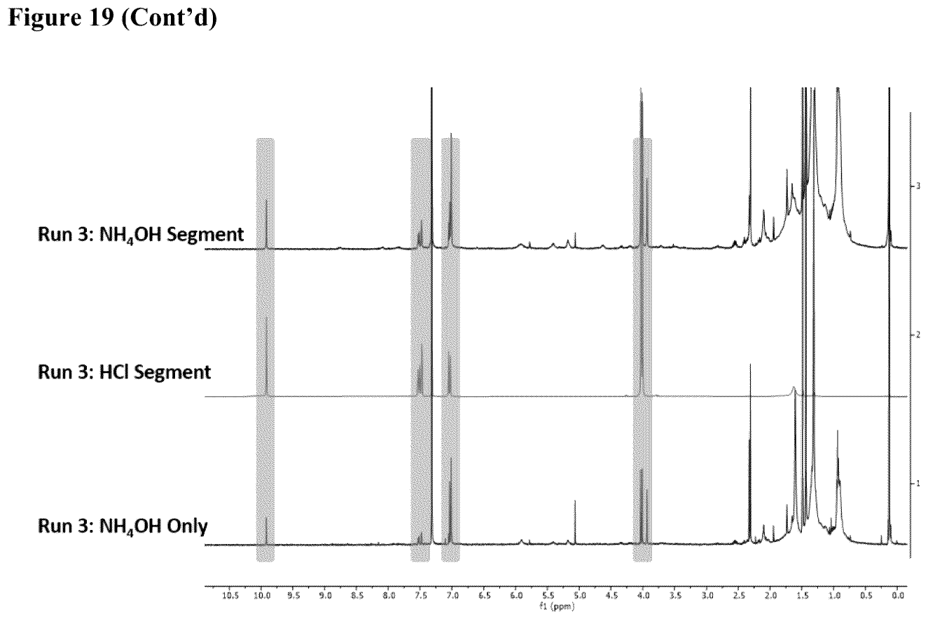

Used resins were treated with 4:1 THF: NH₄OH and agitated by a sonicator for 45 minutes. The resulting solution was decanted, and the resins were washed and sonicated in fresh THE two times and filtered to dryness. A sample of the batch was subjected to the ninhydrin test against unused resins to confirm presence of terminal amine. (FIG. 19)

### Attaching and Removal Model

In order to confirm reuse of the resin with the method of the invention, cleavage conditions were investigated using a model to load on and off the same batches of resins. Conditions were explored using a dimethoxybenzaldehyde model attached on the resin bead. To detach from the polystyrene matrix, an imine hydrolysis must occur to retrieve back the aldehyde and free amine. Usual conditions to achieve hydrolysis use acidic aqueous conditions, however, due to the hydrophobic nature of the polymer, a series of test was performed diluted in THF. A model scheme of the investigation is shown below:

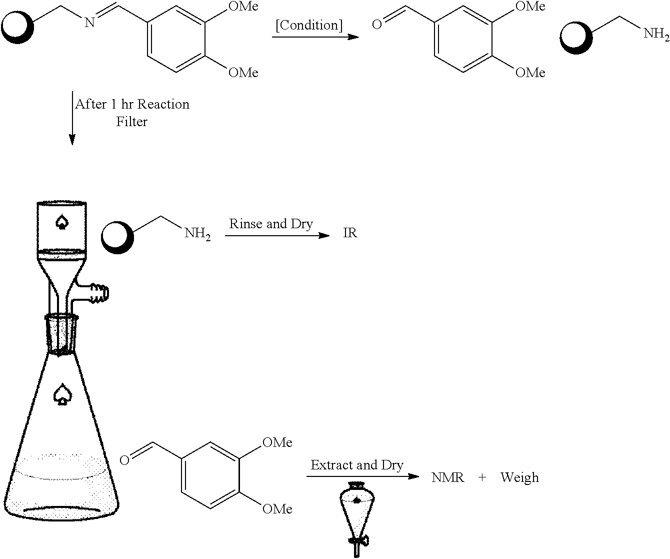

Three separate batches starting at 100 mg of resins loaded with dimethoxybenzyl were introduced in three separate conditions: 1) 1 M HCl/THF (1:4, 5 ml), 2) NH₄OH/THF (1:4, 5 ml), and 3) 1 M HCl/THF (1:4, 5 ml) then replaced by NH₄OH/THF (1:4, 5 ml). Samples were mechanically agitated for 2 hours total. Once finished, the resins were filtered and dried and the filtrate were extracted. The same recovered resins were re-subjected to reloading of model and detachment for another two times to check on mass recovery and loading capacity over repeated use.

The filtrate was extracted and confirmed with NMR to show acidic conditions return quantitative mass crude with minimal contamination while NH₄OH featured only trace amounts with significant peaks in grease region. Continuous runs of resin show with NMR (FIG. 19) more lessened grease peaks over time with HCl, but continues to be present in NH₄OH washes, with the exclusive basic wash consistently having less grease than the combined washes, suggesting a possible need for alternative basic wash to facilitate integrity of the resin for longer periods of time. However, the loading sizes remained relatively consistent over all three samples.

Material recovery was also studied. Conditions 2 and 3 during the run trial were weighed before and after each loading and separation steps (Table 3). The acidic component of condition 3 was able to release the majority of the initially loaded model. Mass loss in the resin over runs is due to removal of resin for sampling.

TABLE 3
Mass recovery per trial run of dimethoxybenzyl model resin test.

| | Run #1 | Run #2 | | | Run #3 | | |
|---|---|---|---|---|---|---|---|
| | ^{A} Resin | ^{A} Resin | | | ^{A} Resin | | |
| | mass | mass | | | mass | ^{B} Recovered | |
| | (Δmass) | (Δmass) | ^{B} Recovered P | | (Δmass) | mass | |
| Method: | (mg) | (mg) | (mg) | ^{C} % | (mg) | (mg) | ^{C} % |
| 2 Only | 103 (14.2) | 63.3 (8.7) | 1.9 ^{E} | <22% | 39 (12.3) | 5.9 ^{E} | <50% |
| NH₄OH | | | | | | | |
| 3 HCl □ | 106 (14.5) | 34.2 (4.7) | 4.7 ^{D} | >95% | 12.3 (2.3) | 2.2 ^{D} | 95% |
| NH₄OH | | | | | | | |

^{A} initial total resin mass with Dimethoxybenzyl attached (mass of just Dimethoxybenzyl attached)
^{B} crude mass retrieved by extraction
^{C} Total crude mass percent recovery compared to attached Dimethoxybenzyl model
^{D} Significant amount of P found in crude observed in NMR of HCl Extraction component
^{E} majority of grease found in crude observed in NMR.

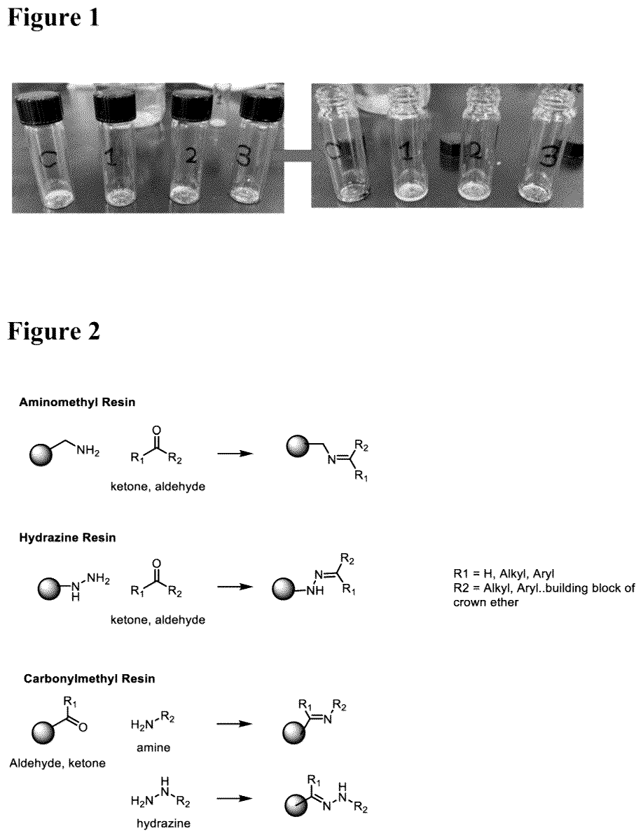

As seen in FIG. 1, the resin component was tested by ninhydrin test along with commercial sample to observe the presence of free amine. Resins subjected to first acid then basic conditions suggested both may be preferred to detach the loaded reagent and then neutralize the amine to become active again. IR was also used to compare with fresh commercial control and found identical results with the combined treatment.

### Synthesis of Model Precursors for Optimal Electrophilic/Nucleophilic Placement

In experimental studies, ring closure with a nucleophile being loaded on the resin and electrophilic tosylated-PEG in solution have been performed. A reverse of that reaction may be performed to assess whether cyclization may occur with a free floating nucleophile. A reaction scheme of the reverse reaction for model precursors is provided below:

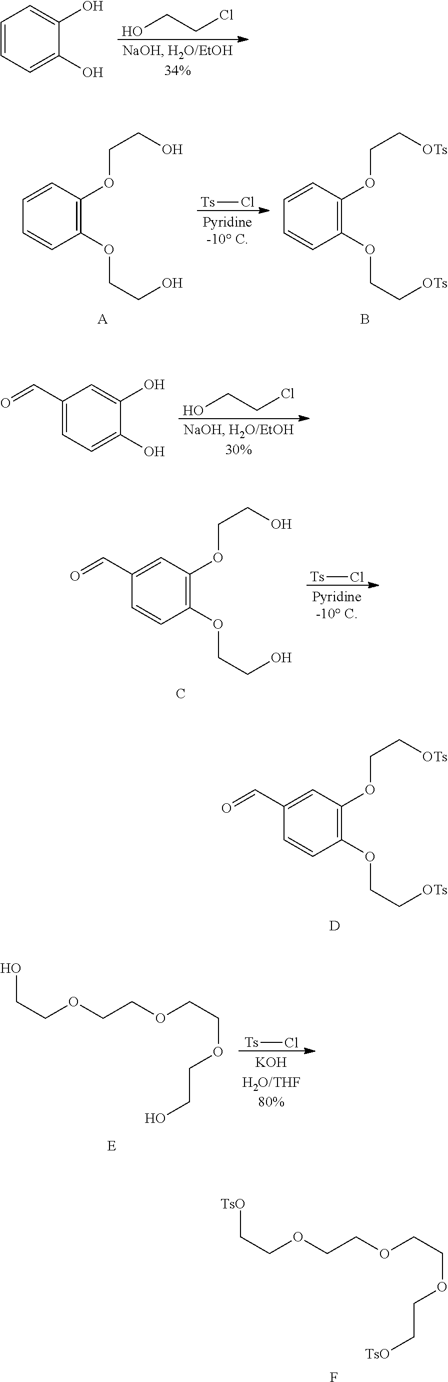

An overview of studies which may be conducted with the model precursors is provided below:

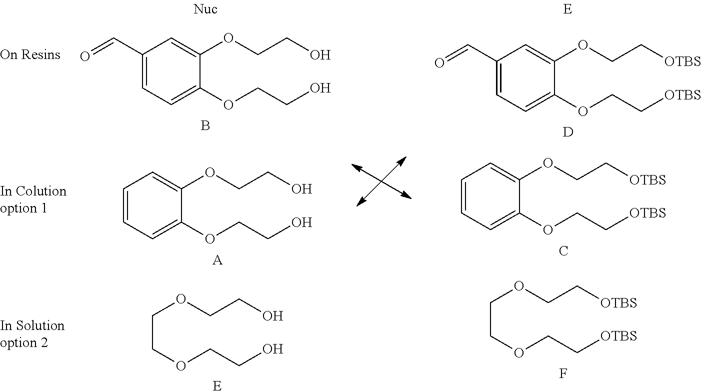

Possible combinations to compare from this set:
B-C, B-F
A-D, E-D

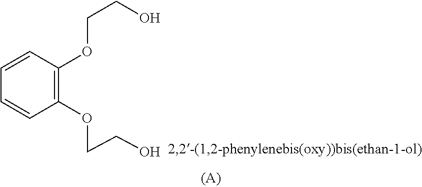 [SMILES: OCCOc1ccccc1OCCO]

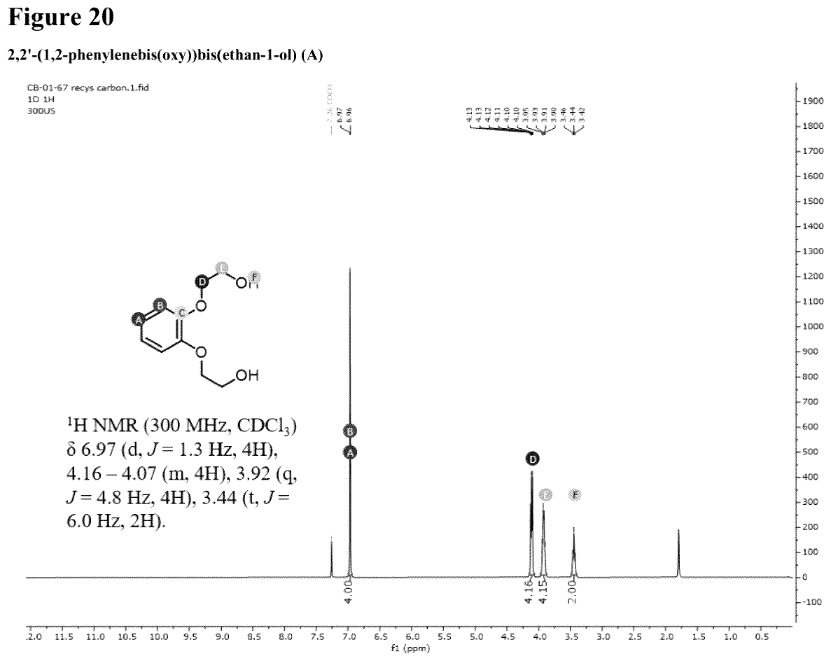

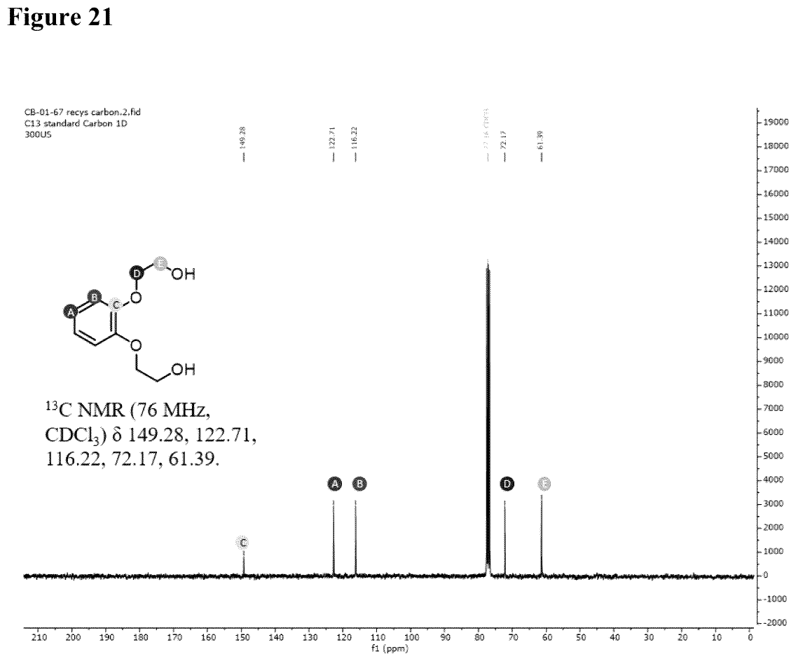

Under argon, catechol (5.5 g, 50 mmol) was dissolved in 95% ethanol (50 ml) and placed in an ice bath. A solution of NaOH (5 g in 3:1 ethanol:water, 50 ml) was added in slowly, following dropwise addition of 2-chloroethanol (10 ml). The mixture was refluxed for 48 hr, then evaporated the ethanol via vaccou. The brown crude was washed with water (20 ml) and extracted by DCM (3×20 ml). The organic solution was collected, dried with MgSO₄, filtered and concentrated. The titled compound was purified by recrystallization of ethanol to produce white solid that could be filtered and dried. Characterization matched reported literature. Yield: 34% Melting point: 72-74° C. ¹H NMR (301 MHZ, CDCl3) δ6.97 (s, 4H), 4.12 (ddd, J=5.4, 3.0, 1.3 Hz, 4H), 4.01-3.85 (m, 4H), 3.49 (t, J=6.1 Hz, 2H). ¹³C NMR (76 MHz, CDCl3) δ 149.28, 122.71, 116.22, 72.17, 61.39.
HRMS (ESI) [M+H]⁺ (C₁₀H₁₅O₄⁺)=calc'd 199.09649, found 199.09474. See FIGS. 20 and 21.

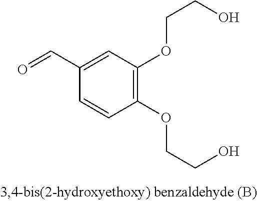 [SMILES: O=Cc1ccc(OCCO)c(OCCO)c1]

Follows the same procedure to compound A. After recrystallization from ethanol, the titled compound was obtained as a white solid.

The following crown ethers were prepared using the method of the invention:

### 4′-Formylbenzo[18C6]

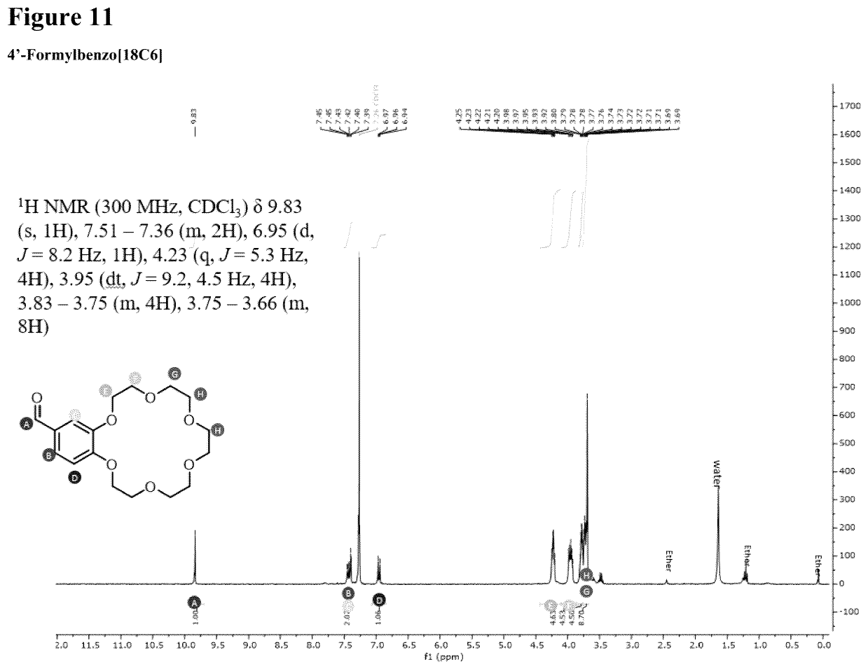

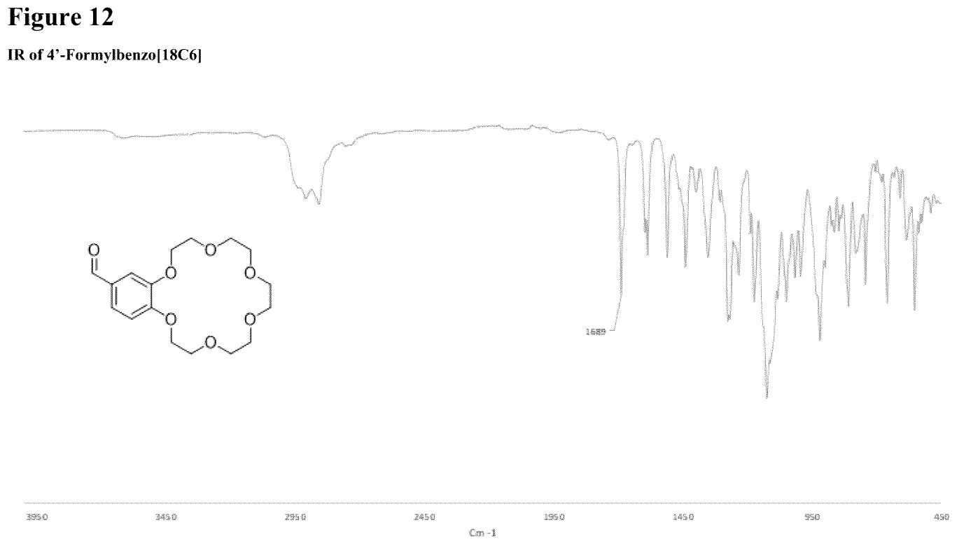

Compound is a fluffy white solid. Rf: 0.38-0.44 in 2 Hexane: 1 DCM: 1 Acetone. ¹H NMR (300 MHz, CDCl₃) δ 9.83 (s, 1H), 7.51-7.36 (m, 2H), 6.95 (d, J=8.2 Hz, 1H), 4.23 (q, J=5.3 Hz, 4H), 3.95 (dt, J=9.2, 4.5 Hz, 4H), 3.83-3.75 (m, 4H), 3.75-3.66 (m, 8H). See FIGS. 11 and 12.

### 4′-Formylbenzo[21C7]

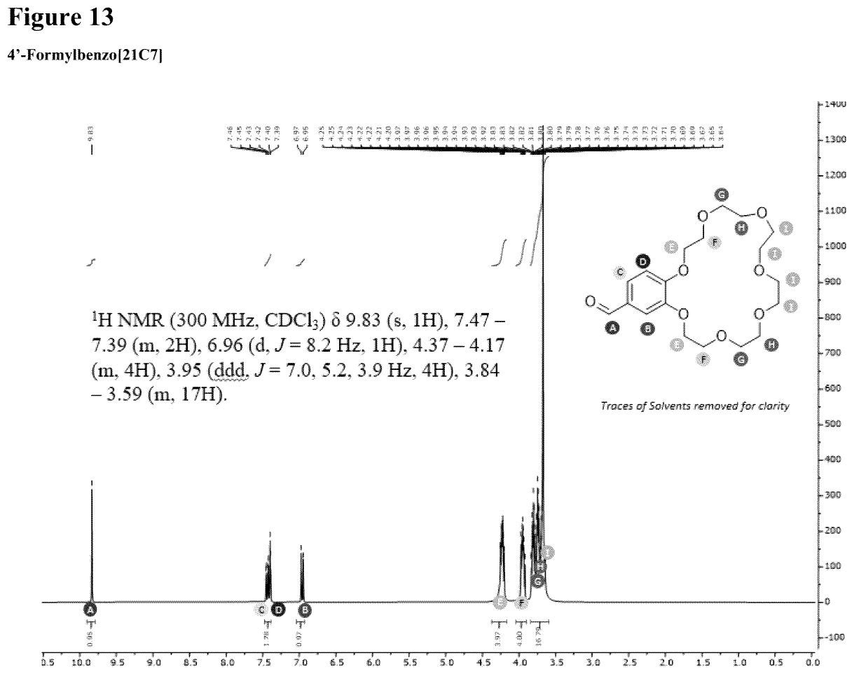

Compound can be a fluffy white solid if complexed with metal or yellow oil uncomplexed. Rf: 0.38-0.44 in 2 Hexane: 1 DCM: 1 Acetone. ¹H NMR (300 MHZ, CDCl₃) δ9.83 (s, 1H), 7.47-7.39 (m, 2H), 6.96 (d, J=8.2 Hz, 1H), 4.37-4.17 (m, 4H), 3.95 (ddd, J=7.0, 5.2, 3.9 Hz, 4H), 3.84-3.59 (m, 17H). See FIG. 13.

### 4′-Formylbenzo[24C8]

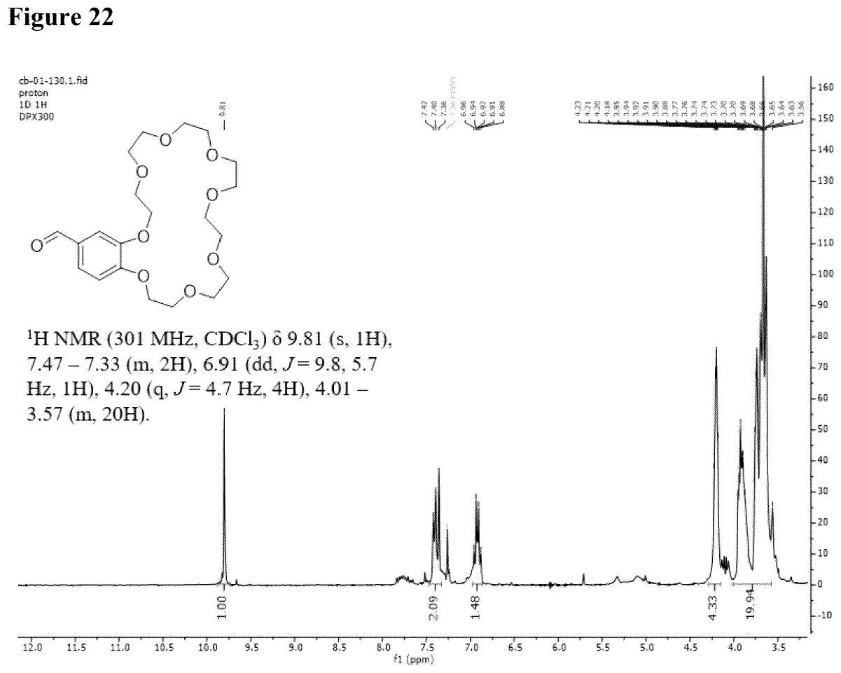

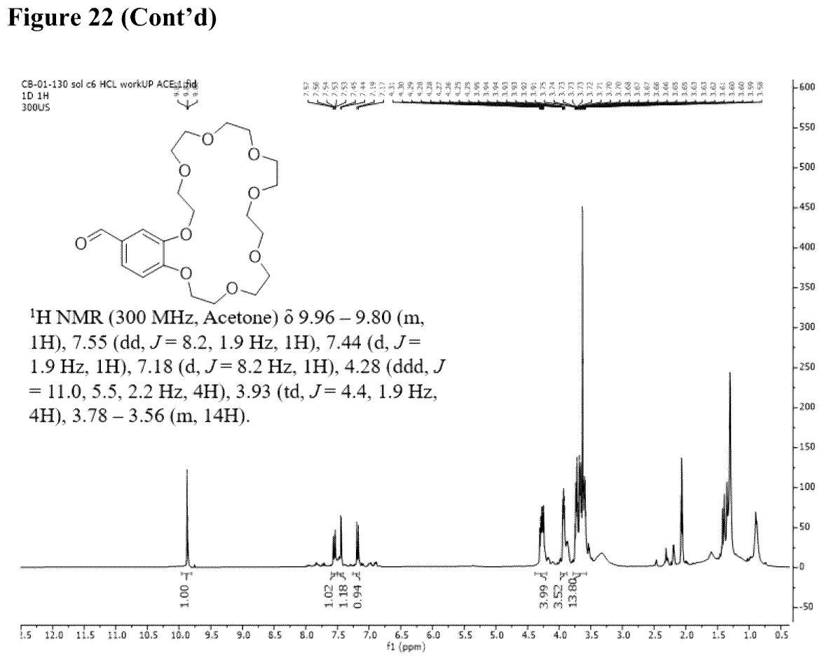

¹H NMR (301 MHz, CDCl₃) δ 9.81 (s, 1H), 7.47-7.33 (m, 2H), 6.91 (dd, J=9.8, 5.7 Hz, 1H), 4.20 (q, J=4.7 Hz, 4H), 4.01-3.57 (m, 20H). ¹H NMR (300 MHz, d⁶-Acetone) δ9.87 (s, 1H), 7.55 (dd, J=8.2, 1.9 Hz, 1H), 7.44 (d, J=1.9 Hz, 1H), 7.18 (d, J=8.2 Hz, 1H), 4.28 (ddd, J=11.0, 5.5, 2.2 Hz, 4H), 3.93 (td, J=4.4, 1.9 Hz, 4H), 3.78-3.56 (m, 14H). HRMS (ESI) C₂₁H₃₃O₉⁺ [M+H]⁺=Observed: m/z 429.2121, Theoretical: m/z 429.2119. C₂₁H₃₂O₉Na⁺ [M+Na]⁺=Observed: m/z 451.1939, Theoretical: m/z 451.1939. LC-LRMS: 429.9 (M+H⁺), 446.9 (M+NH₄⁺). See FIG. 22.

### 4′-Formylbenzo[27C9]

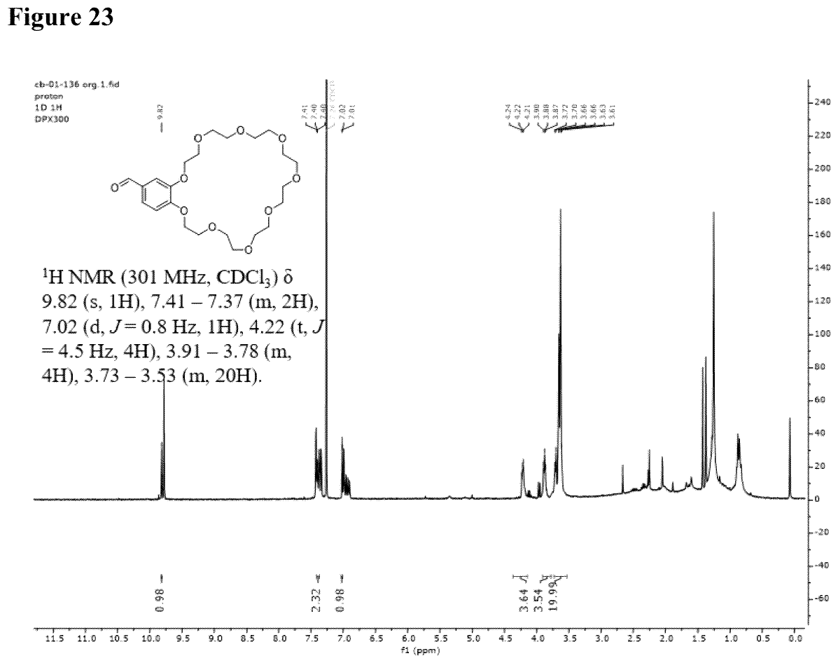

¹H NMR (301 MHz, CDCl₃) δ 9.82 (s, 1H), 7.41-7.37 (m, 2H), 7.02 (d, J=0.8 Hz, 1H), 4.22 (t, J=4.5 Hz, 4H), 3.91-3.78 (m, 4H), 3.73-3.53 (m, 20H). HRMS (ESI) C₂₃H₃₆O₁₀[M+H]⁺=Observed: m/z 473.2378, Theoretical: m/z 473.2381 C₂₃H₃₆O₁₀Na⁺ [M+Na]⁺=Observed: m/z 495.2199, Theoretical: m/z 495.2201. C₂₃H₃₆O₁₀K⁺ [M+K]⁺=Observed: m/z 511.1936, Theoretical: m/z 511.1940. LC-LRMS: 490.1 (M+NH₄⁺), 495.2 (M+Na⁺), 511 (M+K⁺). See FIG. 23.

### 4-Formylbenzo[30C10]

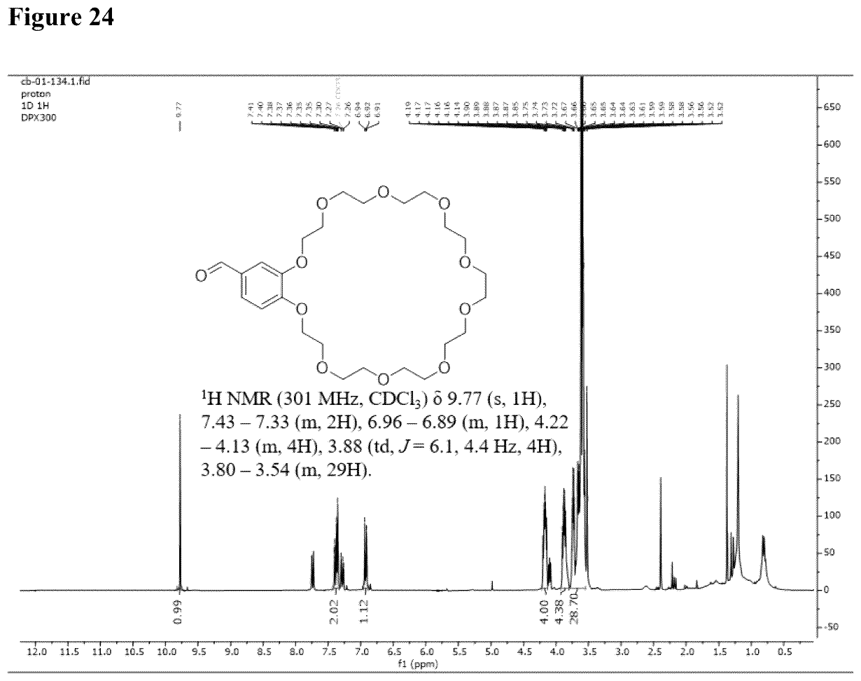

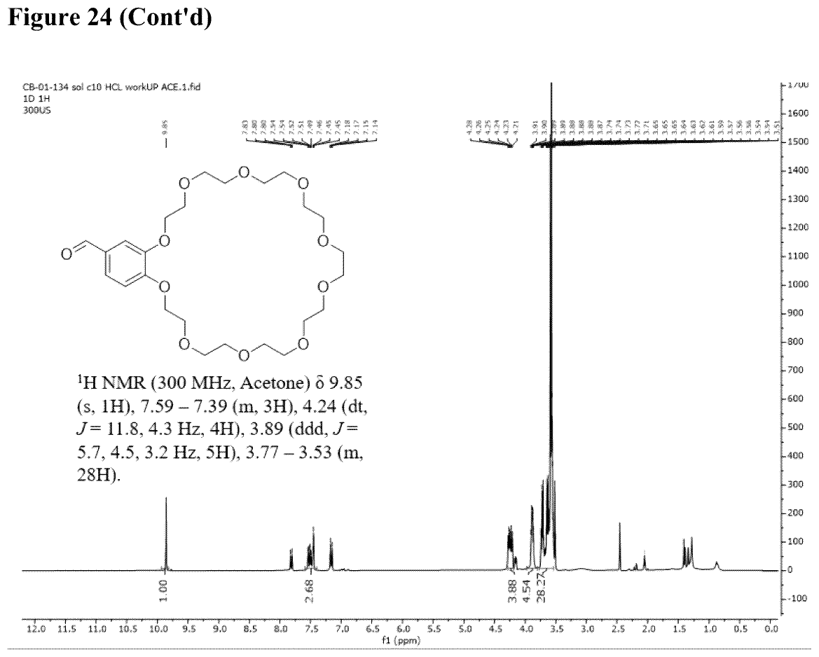

Solution Method. Conversion: 91.6%
Resin Method. Yield: 62.6%
The compound was not purified. 1H NMR (301 MHz, CDCl3) δ 9.77 (s, 1H), 7.43-7.33 (m, 2H), 6.96-6.89 (m, 1H), 4.22-4.13 (m, 4H), 3.88 (td, J=6.1, 4.4 Hz, 4H), 3.80-3.54 (m, 29H). 1H NMR (300 MHz, d6-Acetone) δ9.85 (s, 1H), 7.59-7.39 (m, 3H), 4.24 (dt, J=11.8, 4.3 Hz, 4H), 3.89 (ddd, J=5.7, 4.5, 3.2 Hz, 5H), 3.77-3.53 (m, 28H). HRMS (ESI) C25H40011[M+H]+=Observed: m/z 517.2641, Theoretical: m/z 517.2643 C25H40011Na+ [M+Na]+=Observed: m/z 539.2457, Theoretical: m/z 539.246. LC-LRMS: 297.4 (M+2K+). See FIG. 24.

In a separate study, a number of different conditions were used for generating the aryl aldehyde 18C6 derivatives using compound 1e employing 1 or 1.05 equivalents of the di-electrophile. Provided below is a table summarizing the results of the study:

| | Resin brand | | | | | CE abundance |
|---|---|---|---|---|---|---|
| | with loaded | | | Other | Time ^{b} | in crude |
| Entry | SM ^{a} | Base, eq | Solvent, ml | additions | (h) | (%) ^{c} |
| 1 | Sigma 70-90 | NaOH, 1.25 | 1:1 PhMe:H₂O | TBAI | 48 | 16% |
| | 1-1.5 mmol/g | | 4 ml | | | |
| 2 | Sigma 70-90 | K₂CO₃, 3 | 1:1 MeCN:THF | | 48 | 76% |
| | 1-1.5 mmol/g | | 10 ml | | | |
| 3 | Sigma 70-90 | NaH, 3 | 1:1 MeCN:THF | | 48 | 43% |
| | 1-1.5 mmol/g | | 10 ml | | | |
| 4 | Sigma 70-90 | KHCO₃, 2.5 | DMF 5 ml | | 48 | 9% |
| | 1-1.5 mmol/g | | | | | |
| 5 | Sigma 70-90 | K₂CO₃, 3 | 1:5 MeCN:THF | | 48 | 62% |
| | 1-1.5 mmol/g | | 10 ml | | | |
| 6 | Sigma 70-90 | K₂CO₃, 3 | 1:1 MeCN:THF | NaI | 48 | 16% |
| | 1-1.5 mmol/g | | 10 ml | | | |
| 7 | Sigma 70-90 | K₂CO₃, 3 | 1:1 DMF:THF | | 168 | 83% |
| | 1-1.5 mmol/g | | 10 ml | | | |
| 8 | Sigma 70-90 | K₂CO₃, 3 | 1:1 DMF:MeCN | | 72 | 59% |
| | 1-1.5 mmol/g | | 10 ml | | | |
| 9 | Sigma 70-90 | K₂CO₃, 3 | DMF 10 ml | | 72 | 36% |
| | 1-1.5 mmol/g | | | | | |
| 10 | Sigma 70-90 | K₂CO₃, 3 | MeCN 10 ml | | 72 | 55% |
| | 1-1.5 mmol/g | | | | | |
| 11 | Sigma 70-90 | K₂CO₃, 3 | THF 10 ml | | 72 | 32% |
| | 1-1.5 mmol/g | | | | | |
| 12 | Aaptec | K₂CO₃, 3 | MeCN 10 ml | | 72 | 72% |
| 13 | ChemImpex | K₂CO₃, 3 | MeCN 10 ml | | 72 | 12% |
| 14 | Sigma 100-200 | K₂CO₃, 3 | THF 10 ml | | 72 | 41% |
| 15 | ChemImpex | K₂CO₃, 3 | THF, 10 ml | | 72 | 41-44% |
| 16 | ChemImpex | K₂CO₃, 3 | THF, 10 ml | | 144 | 63% |

^{a} SM refers to 3,4-dihydroxybenaldehyde that is loaded unto the resin. The scale is either 0.21 or 0.24 mmol of SM.
^{b} refluxing time at boiling point of solvents.
^{c} percentage was determined by NMR through integration between all aldehyde signals of products and unreacted 3,4-dihydroxybenzaldehyde in acetone-d₆.
N.R = No reaction, starting material is obtained unchanged

While the invention has been described with reference to preferred embodiments, the invention is not or intended by the applicant to be so limited. A person skilled in the art would readily recognize and incorporate various modifications, additional elements and/or different combinations of the described components consistent with the scope of the invention as described herein.

We claim:

1. A method for preparing a crown ether, the method comprising: loading a resin having an amino functional group with a linking compound having a carbonyl selected for forming an imine bond with the amino functional group, the linking compound further comprising at least two carbon atoms each having an optionally protected hydroxyl group; coupling the linking compound to a polyether polyol having two terminal carbon atoms each bonded to an optionally protected hydroxyl group to obtain a cyclized intermediate, whereby each said at least two carbon atoms forms an ether bond with an associated one of the two terminal carbon atoms; and removing the cyclized intermediate from the resin to obtain the crown ether.

2. The method of claim 1, wherein the linking compound comprises a benzene compound, the at least two carbon atoms forming two carbon ring atoms of the benzene compound.

3. The method of claim 2, wherein the carbonyl forms part of a formyl bonded to the benzene compound, the formyl being located para to the optionally protected hydroxyl group of one said two carbon ring atoms.

4. The method of claim 2, wherein the at least two carbon atoms form two adjacent carbon ring atoms of the benzene compound, the polyether polyol comprising polyethylene glycol.

5. The method of claim 4, wherein the polyethylene glycol has the chemical formula H—(O—CH₂—CH₂)ₙ—OH, wherein n is an integer between 2 and 18, and terminal hydroxyl groups of the polyethylene glycol are optionally protected.

6. The method of claim 5, wherein n is an integer between 5 and 9.

7. The method of claim 1, wherein the hydroxyl groups of the at least two carbon atoms are unprotected, and the hydroxyl groups of the two terminal carbon atoms are protected, optionally wherein the hydroxyl groups of the two terminal carbon atoms are protected with methanesulfonyl or toluenesulfonyl.

8. The method of claim 1, wherein the linking compound is 3,4-dihydroxybenzaldehyde.

9. The method of claim 1, wherein the resin comprises an aminomethylpolystyrene resin, optionally wherein the aminomethylpolystyrene resin is crushed.

10. The method of claim 1, wherein said coupling the linking compound to the polyether polyol is performed with a base, optionally wherein the base is K₂CO₃.

11. A method for preparing a crown ether, the method comprising: loading a resin having a resin functional group with a linking compound having a functional group selected for coupling to the resin functional group and at least two carbon atoms each having an optionally protected hydroxyl group; coupling the linking compound to a polyether polyol having two terminal carbon atoms each bonded to an optionally protected hydroxyl group to obtain a cyclized intermediate, whereby each said at least two carbon atoms forms an ether bond with an associated one of the two terminal carbon atoms; and removing the cyclized intermediate from the resin to obtain the crown ether.

12. The method of claim 11, wherein the resin functional group is an amino functional group, an aminomethyl functional group, a hydrazinyl functional group, a carbonylmethyl functional group, a hydroxymethyl functional group, a methanethiol functional group, an ethane-1,2-diol functional group, a maleimide functional group or a furanyl functional group.

13. The method of claim 11, wherein the functional group comprises a carbonyl, carboxyl, thiol, ketone, aldehyde, amine, hydrazine, boronic acid or maleimide.

14. The method of claim 11, wherein the linking compound comprises a substituted aryl or heteroaryl compound.

15. The method of claim 14, wherein the at least two carbon atoms form two carbon ring atoms of the substituted aryl or heteroaryl compound.

16. The method of claim 15, wherein the substituted aryl or heteroaryl compound comprises a benzene compound, the functional group being located para to the optionally protected hydroxyl group of one said two carbon ring atoms.

17. The method of claim 14, wherein the at least two carbon atoms form two adjacent carbon ring atoms of the substituted aryl or heteroaryl compound, the polyether polyol comprising polyethylene glycol.

18. The method of claim 17, wherein the polyethylene glycol has the chemical formula H—O—CH₂—CH₂)ₙ—OH, wherein n is an integer between 5 and 9, and terminal hydroxyl groups of the polyethylene glycol are optionally protected.

19. The method of claim 11, wherein the hydroxyl groups of the at least two carbon atoms are unprotected, and the hydroxyl groups of the two terminal carbon atoms are protected, optionally wherein the hydroxyl groups of the two terminal carbon atoms are protected with methanesulfonyl or toluenesulfonyl.

20. The method of claim 11, wherein the hydroxyl groups of the at least two carbon atoms are protected, and the hydroxyl groups of the two terminal carbon atoms are unprotected, optionally wherein the hydroxyl groups of the at least two carbon atoms are protected with methanesulfonyl or toluenesulfonyl.

21. The method of claim 11, wherein the resin comprises an aminomethylpolystyrene resin, optionally wherein the aminomethylpolystyrene resin is crushed.

22. The method of claim 11, wherein said coupling the linking compound to the polyether polyol is performed with a base, optionally wherein the base is K₂CO₃.

23. A method for preparing a crown ether, the method comprising:
loading a resin having a resin functional group with a linking compound having a functional group selected for coupling to the resin functional group and at least two carbon atoms each substituted with a first substituent;
coupling the linking compound to a reactant compound of the formula X—CH₂—CH₂ (O—CH₂—CH₂)ₙ—X, wherein n is an integer between 1 and 17, and X is a second substituent, one of the first and second substituents being a leaving group and a remaining one of the first and second substituents being a nucleophilic substituent, whereby the linking compound and the reactant compound undergo a substitution reaction with the nucleophilic substituent replacing the leaving group to thereby obtain a cyclized intermediate; and
removing the cyclized intermediate from the resin to obtain the crown ether.

24. The method of claim 23, wherein the first substituent is the nucleophilic substituent, and the second substituent is the leaving group.

25. The method of claim 23, wherein the first substituent is the leaving group, and the second substituent is the nucleophilic substituent.

26. The method of claim 23, wherein the leaving group is selected from the group consisting of tosylate, mesylate, bromide, iodide, triflate and chloride.

27. The method of claim 23, wherein the nucleophilic substituent is hydroxyl.

28. The method of claim 23, wherein the resin functional group is an amino functional group, an aminomethyl functional group, a hydrazinyl functional group, a carbonylmethyl functional group, a hydroxymethyl functional group, a methanethiol functional group, an ethane-1,2-diol functional group, a maleimide functional group or a furanyl functional group.

29. The method of claim 23, wherein the functional group comprises a carbonyl, carboxyl, thiol, ketone, aldehyde, amine, hydrazine, boronic acid or maleimide.

30. The method of claim 23, wherein the linking compound comprises a substituted aryl or heteroaryl compound, the at least two carbon atoms forming two adjacent carbon ring atoms of the substituted aryl or heteroaryl compound.

31. The method of claim 30, wherein the substituted aryl or heteroaryl compound comprises a benzene compound, the functional group being located para to the first substituent of one said two adjacent carbon ring atoms.

32. The method of claim 23, wherein n is an integer between 4 and 8.

33. The method of claim 23, wherein the resin comprises an aminomethylpolystyrene resin, optionally wherein the aminomethylpolystyrene resin is crushed.

34. The method of claim 23, wherein said coupling the linking compound to the reactant compound is performed with a base, optionally wherein the base is K₂CO₃.
